(19) United States
(12) Patent Application Publication
(10) Pub. No.: US 2026/0130847 A1
(43) Pub. Date: May 14, 2026

(54) INGESTIBLE IN VIVO-ASSEMBLING DRUG RELEASE FORMULATIONS AND METHODS

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Gary W. Liu, Cambridge, MA (US); Matthew Pickett, Canton, CT (US); Robert S. Langer, Newton, MA (US)

(73) Assignees: Massachusetts Institute of Technology, Cambridge, MA (US); The Brigham and Women's Hospital, Inc., Boston, MA (US)

(21) Appl. No.: 19/120,005

(22) PCT Filed: Oct. 12, 2023
(86) PCT No.: PCT/US2023/076701

§ 371 Date: Apr. 10, 2025

Related U.S. Application Data
(60) Provisional application No. 63/415,366, filed on Oct. 12, 2022.

(51) Int. Cl. A61K 9/00; A61K 9/06; A61K 38/47; A61K 47/02; A61K 47/10; A61K 47/36

(52) U.S. Cl. CPC A61K 9/0065; A61K 9/0095; A61K 9/06; A61K 38/47; A61K 47/02; A61K 47/10; A61K 47/36; C12Y 302/01108

(57) ABSTRACT

Systems and methods related to drug delivery are provided. In one arrangement, a fluid is administered to a subject in drinkable form, which can partially or fully solidify in the stomach or another area of the gastrointestinal tract to form a drug release article or composition.

### TECHNICAL FIELD

Systems and methods related to drug release are generally described.

### BACKGROUND OF THE INVENTION

Drug delivery is a field that has benefited from significant research, development and commercialization. One characteristic of drug delivery is that oral delivery can be difficult, unpleasant, or impossible for some subjects, especially oral delivery of non-fluid drugs, for example, pills, gel tabs, or capsules. Oral solid drug dosage forms such as tablets and capsules are a cornerstone of medicine, but present challenges to some patients, typically older and younger patients, for these reasons. Fluid ingestible drugs (e.g., drinkable drug formulations) are useful in many, but not in all circumstances. While liquid drug formulations are easier to swallow, they sometimes lack the capacity to localize therapeutics and excipients and to provide ideal release profiles. For example, in some cases, they may exhibit shorter gastric residence times compared to solids.

For these and other reasons, the inventors of this disclosure have developed ways of changing the state of a drug release formulation after administration to a subject.

### SUMMARY

Systems and methods related to drug release are generally described. This disclosure provides one or more inventions that are outlined in general in the claims below. Other aspects are presented in the description prior to the claims.

Some aspects are related to compositions.

In some embodiments, the composition comprises a first pharmaceutically acceptable fluid and a second pharmaceutically acceptable fluid, wherein the first and second pharmaceutically acceptable fluids are configured to polymerize to form a double network hydrogel when mixed, and wherein the first and second pharmaceutically acceptable fluids are suitable for oral administration to a subject. In some embodiments, the wherein the first pharmaceutically acceptable fluid of the composition comprises at least two crosslinkers and the second pharmaceutically acceptable fluid of the composition comprises at least two hydrogel precursors. Some aspects are related to kits. In some embodiments, the kit comprises at least 200 mL of the first pharmaceutically acceptable fluid of the composition and at least 20 mL of the second pharmaceutically acceptable fluid of the composition. In some embodiments, the composition comprises a fluid drinkable by a subject, formulated to thicken to form a solid excipient when exposed to conditions similar or identical to those of at least one portion of the gastrointestinal tract of the subject. In some embodiments, the composition comprises a first configuration having a first, non-solid viscosity and formulated to thicken to a second configuration having a second viscosity greater than the first viscosity when exposed to conditions similar or identical to those of at least one portion of the gastrointestinal tract of a subject. In some embodiments, the composition comprises a hydrogel that undergoes a transition from a liquid-to-solid state within the stomach and comprises two distinct polymer networks.

Some aspects are related to methods.

In some embodiments, the method comprises forming a double network hydrogel at a location internal to a subject. In some such embodiments, the method comprises orally administering a first pharmaceutically acceptable fluid and a second pharmaceutically acceptable fluid at a location internal of a subject. In some embodiments, the method comprises forming a double network hydrogel within a gastrointestinal tract of a subject, comprising. In some such embodiments, the method comprises polymerizing at least a portion of first pharmaceutically acceptable fluid and at least a portion of second pharmaceutically acceptable fluid within the gastrointestinal tract the subject. In some embodiments, the method comprises drinking a fluid and allowing the fluid to at least partially harden internally of the gastrointestinal tract to form an excipient. In some embodiments, the method comprises drinking a first fluid, drinking a second fluid, and allowing the first and second fluids to at least partially harden internally of the gastrointestinal tract to form an excipient. In some embodiments, the method comprises ingesting a first fluid comprising a crosslinker, after ingesting the first fluid, ingesting a second fluid comprising a hydrogel, and forming discrete hydrogels having an average maximum dimension of less than or equal to 10 cm. In some embodiments, the method comprises ingesting a first fluid comprising a hydrogel precursor, after ingesting the first fluid, ingesting a second fluid comprising a crosslinker, and forming discrete hydrogels having an average maximum dimension of less than or equal to 10 cm.

Some aspects are related to kits.

In some embodiments, the kit is for delivery of a substance to a subject. In some such embodiments, the kit comprises a package containing a first composition and a second composition arranged so as not to be homogenously mixed prior to delivery to the subject, the package configured to deliver at least one of the first and second compositions to a subject as a fluid, wherein the first and second compositions are formulated such that when at least one composition is exposed to conditions similar or identical to those of at least one portion of the gastrointestinal tract of the subject, and at least a portion of the first composition is mixed with at least a portion of the second composition, a solid drug-release excipient is formed. In some embodiments, the kit comprises two solutions, wherein the two solutions, when mixed at a location internal to a subject, polymerize to a solid state comprising two distinct polymer networks, wherein a first polymer network comprises an ionic bond and a second polymer network comprises a covalent bond.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic diagram of polymer networks, according to some embodiments.

FIGS. 2A-2C show an overview of LIFT (liquid in situ-forming and tough) hydrogels. FIG. 2A is a schematic showing LIFT hydrogels form within the stomach after oral administration of (1) a 200-mL crosslinker solution comprising CaCl₂) and a dithiol-containing compound, followed by (2) a 20-40 mL polymer solution comprising alginate and 4-arm PEG maleimide. These two solutions (3) mix within the stomach to form a tough double-network hydrogel (4) within the stomach. FIG. 2B is a schematic of the polymers and reagents used to facilitate crosslinking. Materials were selected due to their established safety profiles. Both a poly(ethylene glycol)-dithiol and dimercaptosuccinic acid (DMSA) were investigated as a dithiol crosslinker. FIG. 2C, left, is a schematic showing LIFT hydrogels may act as controlled release depot through encapsulation of water-insoluble drug that gradually dissolves and diffuses from the hydrogel. FIG. 2C, right, is a schematic showing hydrogels facilitates co-encapsulation and co-localization of therapeutic enzymes and excipient (e.g., CaCO₃) that modulate local pH and protect against proteases.

FIGS. 3A-3D show in vitro characterization of LIFT hydrogels. FIG. 3A, left, shows load-strain curves of LIFT hydrogels comprising 0%, 5%, or 10% 4-arm PEG-maleimide crosslinked in CaCl₂)/PEG-dithiol for 20 min, 37° C., 50 RPM. FIG. 2A, right, shows the load at 90% of the different hydrogel compositions; n=4 hydrogels were tested. FIG. 3B shows images of various compositions of hydrogels before and after 90% strain. Scale bar: 5 cm. FIG. 3C shows load-strain curves of LIFT hydrogels formed in various v/v % mixtures of gastric fluid in water containing CaCl₂)/PEG-dithiol. FIG. 3D shows relation kinetics of LIFT hydrogels immersed in a crosslinker bath comprising CaCl₂)/PEG-dithiol at 37° C., as characterized by rheology. *p-value<0.05; **p-value<0.01; ****p-value<0.0001. Bars represent mean±standard deviation.

FIGS. 4A-4E show in vivo characterization of LIFT hydrogels. FIG. 4A shows hydrogel geometries after in vivo formation in pigs. LIFT hydrogels were formed by endoscopic administration of crosslinker solution (200 mM CaCl₂/10 mM PEG-dithiol) followed by polymer solution (0.5% alginate/5% 4-arm PEG-maleimide). Scale bar: 5 cm. FIG. 4B shows x-ray imaging of LIFT hydrogels in pigs throughout time. Shown is representative of n=3 independent experiments. FIG. 4C shows load-strain curves of alginate or LIFT hydrogels after retrieval from pig stomachs. Hydrogels were characterized by 5 cycles of 90% strain. FIG. 4D shows maximum loads experienced by alginate or LIFT hydrogels throughout 5 cycles of 90% strain. FIG. 4E shows images of retrieved alginate or LIFT hydrogels before and after 90% strain. **p-value<0.01; ***p-value<0.001; ****p-value<0.0001. Bars represent mean±standard deviation.

FIGS. 5A-5C show pharmacokinetics of various oral lumefantrine formulations. FIG. 5A shows plasma lumefantrine concentration over time of free lumefantrine and lumefantrine (960 mg) encapsulated in alginate or LIFT hydrogel. For each treatment, n=3 pigs were tested. FIG. 5B shows lumefantrine area under the curve (AUC) of each formulation. FIG. 5C shows maximum observed lumefantrine concentration (Cm) of each formulation. *p-value<0.05; **p-value<0.01. Bars represent mean±standard deviation.

FIGS. 6A-6D show LIFT hydrogel co-encapsulation of CaCO₃ protects lactase activity after oral delivery. FIG. 6A shows lactase activity after hydrogel encapsulation with or without CaCO₃ co-encapsulation and incubation in SGF for 1 h. Absorbances were normalized to that of alginate/CaCO₃. FIG. 6B shows activity of lactase encapsulated in LIFT hydrogels after 1 h in rat. CaCO₃ was administered separately (LIFT+CaCO₃) or co-encapsulated (LIFT/CaCO₃). Absorbances were normalized by hydrogel mass; n=5 rats were tested for each treatment. FIG. 6C shows activity of lactase encapsulated in LIFT hydrogels after 2 h in rat. Absorbances were normalized by hydrogel mass; n=4 or 5 rats were tested for each treatment. FIG. 6D shows activity of lactase encapsulated in LIFT hydrogels after 6 h in pigs. Hydrogels were retrieved from porcine stomach and randomly sampled. Absorbances were normalized by hydrogel mass; shown is representative of n=3 independent experiments. *p-value<0.05; **p-value<0.01; ***p-value<0.001; ****p-value<0.0001. Bars represent mean±standard deviation.

FIG. 7 shows a structure study of LIFT hydrogels formed in vivo. Top: pigs (n=3) were administered crosslinker solution (200 mM CaCl₂)/10 mM DMSA) followed by hydrogel solution (e.g., a hydrogel precursor, 0.5% alginate/5% 4-arm PEG-maleimide). In some experiments, green dye was added for color contrast. Bottom: pigs (n=3) were administered hydrogel precursor followed by crosslinker solution. In some experiments, green dye was added for color contrast. Generally, the administration order crosslinker+hydrogel resulted in consistent “noodle-like” hydrogels, while hydrogel+crosslinker resulted in more heterogenous hydrogel shapes.

FIG. 8 shows in vivo retention of LIFT hydrogels. Pigs (n=3) were administered a solution of crosslinker (200 mM CaCl₂/10 mM PEG-dithiol) followed by polymer (0.5% alginate/5% 4-arm PEG-maleimide) loaded with 20% BaSO₄ to facilitate X-ray imaging. Hydrogels were present within the gastrointestinal tract up to 24 h after administration.

FIG. 9, left, shows load-strain curves of LIFT hydrogels after 90% strain. Hydrogels were formed in vivo by administration of a crosslinker solution (200 mM CaCl₂)/10 mM DMSA) followed by polymer solution (0.5% alginate/5% 4-arm PEG-maleimide). Hydrogels were retrieved 6-8 h after administration. FIG. 9, right, shows images of LIFT hydrogels after 90% strain. Hydrogels recovered a portion of their initial geometry.

FIG. 10 shows lactase activity after exposure to dithiol compounds. Lactase was added to either DMSA or PEG-dithiol at the indicated concentrations and incubated at 37° C., 50 RPM, 20 min. Lactase activity was quantified by addition of ONPG and analysis of the colored product. Absorbance was normalized to an untreated lactase control.

FIG. 11 shows LIFT hydrogels after formation in rats. Rats were orally gavaged with a crosslinker solution (200 mM CaCl₂/10 mM PEG-dithiol) followed by a polymer solution (0.5% alginate/5% 4-arm PEG-maleimide) with or without CaCO₃.

FIGS. 12A-12C show exemplary bioluminescence results. FIG. 12A shows bioluminescence of an E. coli Nissle 1917 strain engineered to be bioluminescent after various incubation times in PBS or simulated gastric fluid (SGF). FIG. 12B shows bioluminescence of bacteria after exposure to SGF, encapsulated in double network hydrogel (GIST), and encapsulated in GIST along with CaCO₃. FIG. 12C shows bioluminescence of bacteria after suspension in CaCO₃, encapsulation in alginate along with CaCO₃, or in double-network hydrogel (GIST/CaCO₃).

FIG. 13 is a plot of the storage and loss moduli of an example solid, according to some embodiments.

### DETAILED DESCRIPTION

One or more inventions are described herein, in which compositions, including therapeutic compositions which may contain one or more active agents, can be delivered more easily to some subjects than via prior delivery vehicles. The invention(s) provides for more facile delivery not only of therapeutic compositions, but delivery any composition, species, agent, or article delivered to a subject that can benefit from the phase change and/or viscosity change described below. These can include diagnostics, nanoparticles for any purpose, and any other agents the delivery of which will be readily understood by those of ordinary skill in the art to benefit from the present invention(s). In this regard at any location here in which delivery of a composition, active agent, product, or the like is described, it is to be understood the invention(s) encompasses delivery of any other such species as noted herein.

While liquid formulations are easier to ingest, they can be susceptible to rapid dilution within the gastrointestinal tract and may not be as amenable to spatially localized drug/excipients, which particularly challenge efforts to orally deliver biological drugs. A system capable of a programmed liquid-to-solid transition within the stomach provided herein bridges many advantages of these two forms. A thickened, or solid matrix facilitates spatial proximity of drug and excipients that can modulate drug release and/or protect drug activity against the harsh gastric environment, and thus augment gastric residence of a drug depot.

In one aspect, the invention involves a fluid administrable to a subject (e.g., drinkable by the subject) which is formulated to at least partially thicken or change in viscosity after administration, so that what is initially administered as a fluid or lower-viscosity material changes in vivo to become a higher-viscosity material, which can become a semi-solid, hydrogel, solid, or the like (wherever one of these terms, such as “thicken,” is used herein, it is to be understood that this term embraces all of these viscosity and/or phase changes). In some such embodiments, the fluid administrable to a subject is a pharmaceutically acceptable fluid. In this manner, a number of benefits can be realized as will be understood readily by those of ordinary skill in the art. One such benefit and one aspect of the invention is lower-viscosity delivery of a therapeutic agent (e.g., as a drinkable fluid) which becomes a higher-viscosity material internally of a subject in the form of one or more active-agent delivery excipients. Thus, benefits such as improvement and/or control of active agent release profile of a higher-viscosity semisolid, gel, or solid can be realized while being more easily administered in the form of a low viscosity fluid (e.g., compared to a conventional solid tablet used to administer, for example, a therapeutic agent).

Other benefits can include changed gastrointestinal (GI) retention, e.g., a thickened composition such as a gel or solid can have a slower gastrointestinal transit than a liquid, which can provide various benefits including different drug release profile (e.g., a longer period of drug release). In this regard, drug release profiles can be realized such as those described in U.S. Pat. No. 10,182,985, which is herein incorporated by reference in its entirety.

Another benefit can include a thickened composition such as a gel or solid being able to protect therapeutic agents like biologics, such as bacteria, peptides, or small molecules (e.g., less than or equal to 1000 Da), as described in more detail elsewhere herein, more effectively than a lower-viscosity (e.g., fluid) delivery/transit/excipient medium. Such protection may be physical in nature, for example, the thickened composition may physically separate an interior of the thickened composition from the exterior of the thickened composition. Thus, the interior of the thickened composition may not have the same conditions as the exterior, which in some cases may be relatively harsh, e.g., acidic in the gastrointestinal tract.

As one related example of how the invention can benefit composition delivery, hydrogels hold great promise for oral delivery of various agents, especially various drugs. However, one challenge is that the gastrointestinal tract is mechanically active, which can physically degrade or damage some ingested materials that are not robust enough to withstand such action. Some hydrogels are adversely affected in this manner. In terms of drug delivery, this may be deleterious as it may break ingested hydrogels and cause leakage of drug from excipients that modulate the drug release and/or protect the drug from degradation by the gastrointestinal tract, thus losing such benefits. As will be apparent to those of ordinary skill in the art in reviewing this disclosure, this challenge is addressed by the present invention(s).

A composition of the invention can be provided it is formulated to thicken to a greater viscosity or hardness, or even to form a solid, when exposed to conditions similar or identical to those of at least one portion of the gastrointestinal tract of the subject. Those of ordinary skill in the art readily understand the range of these conditions. The conditions can be those in vivo, of any aspect of the gastrointestinal tract of any subject that could benefit from the present invention(s), typically a mammal or human subject as described elsewhere herein. These conditions also can be simulated conditions, in vitro. For example, in some cases, the composition may be administered to a subject (e.g., via ingestion), wherein the composition encounters the conditions at the location internal of the subject and thickens to form a product. In some such embodiments, the product comprises a hydrogel, e.g., a double network hydrogel. In some embodiments, the location internal the subject is the GI tract.

In some embodiments, the composition (e.g., a hydrogel) comprises a double network hydrogel. In some embodiments, a double network hydrogel comprises an interpenetrating polymer network comprising at least a first and second interpenetrating polymers. In certain embodiments, the first polymer comprises at least a first cross-link moiety. For example, the interpenetrating polymer network may be formed by mixing two or more monomers (e.g., monomers, oligomers, polymers, and/or prepolymers) and one or more crosslinking reagents (e.g., a bifunctional monomer, a polyfunctional monomer) such that a first monomer reacts forming a first polymer comprising a first crosslink moiety (e.g., comprising at least a portion of a first crosslinking reagent) and/or a second monomer reacts forming a second polymer comprising a second crosslink moiety (e.g., comprising at least a portion of a second crosslinking reagent). In some embodiments, the monomers (e.g., monomers, oligomers, polymers, and/or prepolymers) are biocompatible and/or pharmaceutically acceptable for use at a location internal to the subject.

In one set of embodiments, the monomers (e.g., monomers, oligomers, polymers, and/or prepolymers) and/or the one or more crosslinking reagents are suitable for oral administration to a subject. In some embodiments, the monomers comprise oligomers, polymers, and/or prepolymers and do not need to polymerize at the location internal the stomach.

The term “biocompatible.” as used in reference to a some of the components of the compositions described herein, refers to a chemical or other material that does not invoke a substantial adverse reaction (e.g., deleterious immune response) from an organism or subject (e.g., a mammal), a tissue culture or a collection of cells, or invokes only a reaction that does not exceed an acceptable level.

The term “pharmaceutically acceptable” refers to, within the scope of sound medical judgment, being suitable for use in contact with the tissues of a subject (e.g., a mammal, a human, etc., as described elsewhere herein) without undue toxicity, irritation, and/or allergic response, and being commensurate with a reasonable benefit/risk ratio.

The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig. Additionally, those of ordinary skill in the art will understand the meaning of a location internal to the subject.

As used herein, the term “polymer network” refers to a three-dimensional substance having oligomeric or polymeric strands interconnected to one another by crosslinks. One of ordinary skill will appreciate that many oligomeric and polymeric compounds are composed of a plurality of compounds having differing numbers of monomers. Such mixtures are often designated by the number average molecular weight of the oligomeric or polymeric compounds in the mixture.

The phrase “interpenetrating polymer network,” as used herein, is given its ordinary meaning in the art and generally refers to a polymer network comprising two or more polymer strands in which at least two polymers are at least partially interlaced with one another, such that the network cannot be separated unless chemical bonds are broken. In some embodiments, the at least two polymers interlaced with one another are not (chemically) bonded (e.g., covalently) to each other. In certain embodiments, a first polymer of the at least two polymers interlaced with one another comprises a first crosslinking moiety (e.g., the first polymer is at least partially crosslinked with itself). In some embodiments, a second polymer of the at least two polymers interlaced with one another comprises a second crosslinking moiety (e.g., the second polymer is at least partially crosslinked with itself).

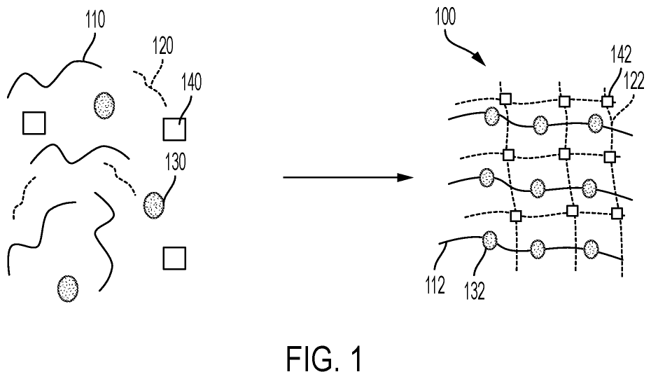

In an exemplary illustrative embodiment, as shown in FIG. 1, polymer network 100 may be formed by the reaction of monomer (e.g., or polymer) 110 with crosslinking reagent (e.g., a first crosslinker) 130 and the reaction of monomer (e.g., or polymer) 120 with crosslinking reagent (e.g., a second crosslinker)140. In some embodiments, polymer network 100 comprises first polymer 112 (e.g., formed from the reaction of monomer 110 and/or crosslinking reagent 130) and second polymer 122 (e.g., formed from the reaction of monomer 120 and/or crosslinking reagent 140) interpenetrating with first polymer 112. In certain embodiments, first polymer 112 comprises a first crosslinking moiety 132 and/or second polymer 122 comprises a second crosslinking moiety 142.

As used herein, the term “crosslink” refers to a connection between two polymer strands, or a connection between two points one a single polymer strand. The crosslink may either be a chemical bond, a single atom, or multiple atoms. The crosslink may be formed by reaction of a pendant group in one polymer strand with the backbone of a different polymer strand, or by reaction of one pendant group with another pendant group. Crosslinks may exist between separate polymer strands and may also exist between different points of the same polymer strand. As used herein, the term “polymer strand” refers to an oligomeric or polymeric chain of one monomer unit, or an oligomeric or polymeric chain of two or more different monomer units. As used herein, the term “prepolymer” refers to oligomeric or polymeric strands which have not undergone crosslinking to form a network.

As used herein, the term “crosslink moiety” or “crosslinking moiety” refers to the bond or atom(s) making up the crosslink between two polymer strands (or between different points on the same polymer strand). In some embodiments, the crosslink moiety comprises one or more chemical bonds, such as an ionic bond, a covalent bond, a hydrogen bond, Van der Waals interactions, and the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. The ionic bond may comprise, for example, a polyvalent cation. Non-limiting examples of polyvalent cations include calcium, barium, strontium, iron, aluminum.

Other polyvalent cations are also possible. In an exemplary embodiment, the polyvalent cation is calcium.

As used herein, the term “crosslinker” or “crosslinking reagent” or “crosslinking agent” refers to a reagent or chemical that reacts and/or interacts with at least two polymer strands to form a crosslinking moiety between the at least two polymer strands.

In some embodiments, the crosslink moiety may be formed by mixing a polymer (or polymer precursor and/or monomer) with a crosslinking agent. Crosslinking agents, in some embodiments, are biocompatible and/or are pharmaceutically acceptable. In some such embodiments, crosslinking reagents may be suitable for oral administration to a subject. In some embodiments, crosslinking agents are FDA-approved and/or generally regarded as safe. Examples of suitable crosslinking agents are described elsewhere herein.

As used herein, the term “hydrogel” refers to a polymer network capable of absorbing a relatively high amount of water (e.g., a high weight percentage of water as compared to the weight of the polymer network e.g., greater than 70 wt % water).

As mentioned above, in some embodiments, the compositions described herein may be formulated to thicken to a greater viscosity or hardness, or even to form a solid, when exposed to conditions similar or identical to those of at least one portion of the gastrointestinal tract of the subject.

Example conditions which may be encountered within the gastrointestinal tract in which the compositions described herein may thicken (e.g., polymerize) include any of a variety of temperatures, solution compositions, and biomaterials. In some cases, a spatially averaged temperature of the gastrointestinal tract in which the composition may be configured to thicken may be greater than or equal to 35 degrees C., greater than or equal to 36 degrees C., greater than or equal to 37 degrees C., or greater than 38 degrees C. In some cases, a spatially averaged temperature of the GI tract may be less than or equal to 39 degrees C., less than or equal to 38 degrees C., less than or equal to 38 degrees C., less than or equal to 37 degrees C., less than or equal to 36 degrees C., or less than or equal to 35 degrees C. Combinations of the foregoing ranges are possible (e.g., greater than or equal to 36 degrees C. and less than or equal to 38 degrees C.). Other ranges are also possible.

In some embodiments, a fluid encountered by the compositions in the GI tract of a subject may be acidic. In some embodiments, a fluid encountered by the compositions in the GI tract of a subject may have a near neutral pH. In some embodiments, the compositions described herein may be configured to thicken (e.g., polymerize) in relatively acidic media. For example, a relatively acidic media may be encountered by the compositions in the stomach of the GI tract, and in some embodiments, the compositions described herein may be configured to thicken in such conditions. In some embodiments, fluid within the GI tract may have a pH of less than or equal to 8, less than or equal to 7, less than or equal to 6, less than or equal to 5, less than or equal to 4, less than or equal to 3, or less than or equal to 2. In some embodiments, fluid within the GI tract may have a pH of greater than or equal to 1, greater than or equal to 2, greater than or equal to 3, greater than or equal to 4, greater than or equal to 5, greater than or equal to 6, or greater than or equal to 7. Combinations of the foregoing ranges are possible (e.g., greater than or equal to 1 and less than or equal to 3). Other ranges are also possible. Note than certain pH ranges may be encountered in certain regions of the GI tract, and those of ordinary skill in the art will recognize the location and general parameters associated with different regions of the GI tract.

In some embodiments, compositions described herein may encounter various biomaterials within the GI tract, including cells, enzymes, and various other proteins, as known to those of ordinary skill in the art. The compositions described herein, as described herein, may thicken in the presence of such biomaterials and may have no significant impact on the function of such biomaterials, for example, within the GI tract. In some embodiments, once thickened, e.g., to form a double network hydrogel, some or all of the biomaterials may not be able to interact with an interior of the double network hydrogel.

Note that the above conditions that may be encountered in the GI tract, in some embodiments, do not encompass the only conditions in which the compositions described herein may thicken. In some embodiments, as described elsewhere herein, the compositions of the present disclosure may be used in and/or at different regions of the body. In some such embodiments, various other conditions may be encountered, wherein the compositions may still be configured to thicken (e.g., polymerize).

Where a lower-viscosity material (e.g., a drinkable fluid) is administered to a subject as described herein, it is to be understood that this material is of a viscosity or hardness lower than that of at least one product resulting from delivery of the material internally of the subject. In one set of embodiments, the difference in viscosity and/or hardness of the material prior to delivery to the subject, as compared to a project resulting internally from that delivery, is at least 10%, or in other environments these 20, 30, 40, 60, or 80% different. And in one set of embodiments the viscosity and/or hardness of the material prior to delivery is compared to at least one product of the material is measured after delivery in the GI tract above the colon, for example, in the stomach. In some such cases, the lower-viscosity material may be administered to a subject and form a product internal to the subject, whereafter the product may be obtained and the viscosity and/or hardness of the product is then measured.

A variety of techniques can be used to thicken or hardened a composition that is introduced to a subject as described herein. In one set of embodiments, a material that is delivered to a subject (optionally one or more species within the material), is selected to thicken when exposed to conditions similar or identical to those internally of the GI tract of the subject. In another set of embodiments, two or more species delivered to the subject react internally to cause thickening. In each case, those of ordinary skill in the art can select materials that are safe for delivery to the subject and that achieve these ends. In all cases, the delivery material and process can be selected such that a desired agent (e.g., a therapeutic agent, diagnostic, etc.) is present in the thickened product internal of the subject after the composition thickens or hardens.

For example, material can be delivered to a subject, optionally from a kit configured for such delivery. In some cases, the kit includes a first composition and a second composition formulated such that when at least one composition is exposed to conditions similar or identical to those of at least one portion of the GI tract of the subject, and at least a portion of the first composition is mixed with at least a portion of the second composition, a thickened product, such as a solid, semi-solid, and/or gel drug-release excipient is formed. As noted, where a “solid,” “semi-solid,” “gel,” or “hydrogel” is used herein, any or all can be used. In a general sense, when “solid” is used herein, this means a material that is one or more of the above (e.g., a product, a solid, semi-solid, gel, or hydrogel) and is thicker and/or of higher viscosity then the material initially delivered to the subject (e.g., a first composition and/or a second composition).

Those of ordinary discussion the art can select first and second compositions to achieve this result. For example, materials that safely thicken in vivo can include those that crosslink via ionic crosslinking. In some cases, materials that safely thicken in vivo may include those that crosslink via covalent bonds (e.g., click chemistry). More is described later in this regard.

The first and second compositions can be mixed prior to delivery to the subject, partially mixed, mixed (or partially mixed) during delivery, or kept separate and administered separately such that they interact first in vivo. Those of ordinary skill in the art can readily provide various kits and methodologies to achieve any such arrangements. For example, a kit may comprise a first and a second composition, wherein the first and second compositions are kept separate (e.g., physically separated in two separate containers) within the kit. In some such embodiments, a subject may ingest the first composition and then subsequently ingest the second composition such that the first and second compositions interact in vivo, e.g., in the gastrointestinal tract of the subject.

According to certain embodiments, a kit is provided. The kit may comprise one or more devices, such as containers or syringes comprising containers (e.g. barrels of the syringes, containers containing a first and/or second composition) that are capable of storing one or more components (e.g., a monomer, a polymer, a crosslinker, a first composition, a second composition, etc.), mixing the one or more components, and/or delivering the one or more components to a tissue site. For example, a dual-barrel syringe, in some embodiments, is capable of storing a first and a second composition separately (e.g., in a first and second barrel) and may facilitate mixing when applying the first and second composition, e.g., at a location internal to a subject.

As noted, in one set of embodiments, the first and/or second compositions may be orally administered, e.g., they may be imbibed by the subject. In another set of embodiments, the first and/or second compositions may be administered non-orally to a location on or in a subject. In this set of embodiments, the compositions may be subcutaneously administered, optionally by injection. Or the compositions may be administered topically to any area external of the subject, or internally at any other location via, e.g., open surgery, minimally-invasive surgery, or the like. For example, compositions can be administered to surfaces and/or internally (e.g., within voids) on or in organs, bones, and/or other body tissues and structures via techniques known to those of ordinary skill in the art or developed subsequently.

The first and second composition may be physically separated before administration, and mixed just prior to or during administration, or after administration has begun. For example, the compositions can be mixed less than 5 minutes prior to administration to a subject, or in other embodiments less than 4 minutes, 3 minutes, 2 minutes, less than one minute, or less than 30 seconds or less than 15 seconds prior to administration. Mixing a short period of time prior to administration can be carried out by any of a variety of techniques known to those of ordinary skill in the art, such as via use of a dual-barrel syringe in which the compositions are kept separate in separate barrels and are mixed just prior to or during expulsion from the syringe, via catheter arrangements, separate administrative delivery pathways, etc.

In some embodiments, the first and second compositions may be mixed during administration, e.g., via a double-barrel syringe which delivers the compositions to the desired location on or in a subject at essentially the same time they are mixed, and/or delivered via separate vehicles (separate tubes, separate syringes, etc.) simultaneously. In other embodiments, one component or composition can be administered to a location on or in a subject, followed by administration of another component or composition where the compositions are mixed at the site of administration.

The kit may comprise any of the compositions described elsewhere herein contained within a first container, which may be conveniently a barrel of a syringe device. In some embodiments, the first container may comprise a first and/or a second composition. In certain embodiments, the first container may further comprise an auxiliary therapeutic agent and/or any other agent desirably delivered along with the compositions described herein. The composition may be in the form of a liquid. In some embodiments, the first container may be a bottle suitable for containing a first composition in a manner than is physically separated from the second composition.

The kit may further comprise a second container containing any of the compositions described elsewhere herein. In some embodiments, the second container may comprise a first and/or a second composition. For example, a syringe device can contain the composition within second barrel/container, e.g., during storage. In some embodiments, the composition may be in the form of a liquid. In some embodiments, the second container may further comprise a therapeutic agent. In some embodiments, the second container may be a bottle suitable for containing a second composition in a manner than is physically separated from the first composition.

As described elsewhere above, administration may comprise any of a variety of suitable methods, in accordance with some embodiments. In some embodiments, administration may comprise mixing of the first and second compositions, which comprises delivering the first and second compositions from the first and second containers to a location internal to the subject.

In one set of embodiments, one of the compositions is provided first in vivo (administered to the subject) in a larger volume and then the second composition is administered in a smaller volume and is formulated to disperse within the first composition, for example forming solid and/or pill or capsule-like articles within the subject. Routine chemistry, including leveraging hydrophilicity, lipophilicity, hydrophobicity, lipophobicity, or other aspects in which the miscibility and/or immiscibility of the first composition relative to the second composition can be controlled, can be used to achieve any such result. In some cases, the first and second compositions may or may not interact with each other in the absence of conditions similar or identical to those of the GI tract of the subject, but such interaction occurs only under such conditions. In other arrangements, the first and second composition interact in this way even in the absence of conditions similar or identical to those of the GI tract. In some instances, the first and second compositions themselves may or may not display a level of miscibility or immiscibility that would result in dispersion of one within the other to form pill or capsule-like entities, but when they interact with each other there is a reaction at their interface, and/or one is caused to react by the other, to cause such dispersion.

For example, in some embodiments, one of the compositions (e.g., a first or a second composition within a kit) may comprise a larger volume than the other composition. In some embodiments, a first of the composition may comprise a volume of greater than or equal to 200 mL, greater than or equal to 225 mL, greater than or equal to 250 mL, greater than or equal to 275 mL, greater than or equal to 300 mL, greater than or equal to 350 mL. In some embodiments, a first of the composition may comprise less than or equal to 400 mL, less than or equal to 350 mL, less than or equal to 300 mL, less than or equal to 275 mL, less than or equal to 250 mL, less than or equal to 225 mL, less than or equal to 200 mL, less than or equal to 175 mL, less than or equal to 150 mL, or less than or equal to 125 mL. Combinations of the foregoing ranges are possible (e.g., greater than or equal to 200 mL and less than or equal to 400 mL). Other ranges are also possible.

In some embodiments, one of the compositions (e.g., a first or a second composition within a kit) may comprise a smaller volume than the other composition. In some embodiments, a second of the composition may comprise a volume of greater than or equal to 20 mL, greater than or equal to 30 mL, greater than or equal to 40 mL, greater than or equal to 50 mL, greater than or equal to 60 mL, or greater than or equal to 70 mL. In some embodiments, a first of the composition may comprise less than or equal to 80 mL, less than or equal to 70 mL, less than or equal to 60 mL, less than or equal to 50 mL, less than or equal to 40 mL, or less than or equal to 30 mL. Combinations of the foregoing ranges are possible (e.g., greater than or equal to 20 mL and less than or equal 25 to 80 mL, greater than or equal to 20 mL and less than or equal to 50 mL). Other ranges are also possible.

In some embodiments, having such different volumes for the first and second composition may not be ideal for mixing the first and second composition. For example, in some embodiments, the first and second compositions may ideally be present in equal concentrations. However, in some embodiments as elsewhere described herein, differing amounts of the compositions may facilitate oral administration, as the large volume of one of the compositions (e.g., the first composition) may be orally administered first and form a “pool” of the composition in the GI tract. In some such embodiments, subsequent oral administration of the other composition (e.g., the second composition) may introduce the second composition in the pool of the first composition and facilitate crosslinking therein. This may dilute and/or avoid interaction between the second composition and a fluid internal to the subject (e.g., gastric fluid). In some embodiments wherein the second composition comprises a therapeutic agent, this may avoid interaction between the therapeutic agent and a fluid internal to the subject, which may prolong the lifetime of the therapeutic agent.

In some embodiments, a combined volume of the first and second compositions may be greater than or equal to 200 mL, greater than or equal to 225 mL, greater than or equal to 250 mL, greater than or equal to 275 mL, greater than or equal to 300 mL, greater than or equal to 350 mL. In some embodiments, a combined volume of the first and second compositions may be less than or equal to 400 mL, less than or equal to 350 mL, less than or equal to 300 mL, less than or equal to 275 mL, less than or equal to 250 mL, less than or equal to 225 mL, less than or equal to 200 mL, less than or equal to 175 mL, less than or equal to 150 mL, or less than or equal to 125 mL. Combinations of the foregoing ranges are possible (e.g., greater than or equal to 200 mL and less than or equal to 400 mL). Other ranges are also possible.

In some embodiments, the each of the first and second compositions may comprise any of a variety of components, as described elsewhere herein. In some embodiments, each of the crosslinkers and/or hydrogel precursors present in the first and/or second composition may be present in any of a variety of suitable amounts. In some embodiments, each of the crosslinkers and/or hydrogel precursors may independently be present in the first and/or second composition in an amount of greater than or equal to 0.01% w/v (e.g., percent weight solute by volume solvent), greater than or equal to 0.1% w/v, greater than or equal to 0.5% w/v, greater than or equal to 1% w/v, greater than or equal to 5% w/v, greater than or equal to 10% w/v, greater than or equal to 20% w/v, or greater than or equal to 30% w/v. In some embodiments, each of the crosslinkers and/or hydrogel precursors may independently be present in the first and/or second composition in an amount of less than or equal to 40% w/v, less than or equal to 30% w/v, less than or equal to 20% w/v, less than or equal to 10% w/v, less than or equal to 5% w/v, less than or equal to 1% w/v, or less than or equal to 0.5% w/v. Combinations of the foregoing ranges are possible (e.g., greater than or equal to 0.01% w/v and less than or equal to 40% w/v). Other ranges are also possible. Those of ordinary skill in the art would be able to select appropriate amounts of each crosslinker and/or hydrogel precursor by performing a quick benchtop gelation test as described elsewhere herein and testing the crosslinking time and/or mechanical properties of the resulting solid (e.g., double network hydrogel).

In some embodiments, each of the first and/or second composition may have a viscosity, e.g., a first viscosity before being mixed. In some embodiments, each of the first and/or second composition may be a fluid, and thus may have a relatively low viscosity. In some embodiments, the viscosity of the first and/or second composition as measured at 25 degrees C. is independently greater than or equal to 0.1 mPa s, greater than or equal to 0.5 mPa s, greater than or equal to 1 mPa s, greater than or equal to 2 mPa s, greater than or equal to 3 mPa s, greater than or equal to 5 mPa s, greater than or equal to 10 mPa s, greater than or equal to 20 mPa s, greater than or equal to 30 mPa s, greater than or equal to 50 mPa s, greater than or equal to 100 mPa s, greater than or equal to 500 mPa s, greater than or equal to 1,000 mPa s, greater than or equal to 5,000 mPa s, greater than or equal to 10,000 mPa s, greater than or equal to 20,000 mPa s, or greater than or equal to 50,000 mPa s. In some embodiments, the viscosity of the first and/or second composition as measured at 25 degrees C. is independently less than or equal to 100,000 mPa s, less than or equal to 50,000 mPa s, less than or equal to 20,000 mPa s, less than or equal to 10,000 mPa s, less than or equal to 5,000 mPa s, less than or equal to 1,000 mPa s, less than or equal to 500 mPa s, less than or equal to 100 mPa s, less than or equal to 50 mPa s, less than or equal to 30 mPa s, less than or equal to 20 mPa s, less than or equal to 10 mPa s, less than or equal to 5 mPa s, less than or equal to 3 mPa s, less than or equal to 2 mPa s, less than or equal to 1 mPa s, or less than or equal to 0.5 mPa s. Combinations of the foregoing ranges is possible (e.g., greater than or equal to 0.1 mPa s and less than or equal to 100,000 mPa s). Other ranges are also possible.

The first and second compositions can interact with each other in a variety of ways, including ionic crosslinking, covalent crosslinking and/or polymerization of monomers and/or oligomers, weak-force crosslinking (H-bonding, van der Waals interactions), or a combination. In one set of embodiments, ionic and/or weak-force crosslinking is used since it is generally more compatible with in vivo use. Polymerization may be possible in other systems, generally not internally of a subject. In some embodiments, a first network and the second network of the double network hydrogel comprise similar types of interactions (e.g., covalent crosslinking). In some embodiments, a first network and the second network of the double network hydrogel comprise different types of interactions, e.g., a first network is covalently crosslinked whereas a second network is ionically crosslinked. Other combinations are also possible.

In some embodiments, as described elsewhere herein, the first and second compositions may be formulated to thicken upon interaction, for example, under conditions similar to those experienced at a location internal to a subject, e.g., within the gastrointestinal tract. In some embodiments, thickening of the first and/or second composition may be due to an interaction between the first and second composition as described above, for example, ionic crosslinking, covalent crosslinking, etc. In some embodiments, such thickening may produce a double network hydrogel. In some embodiments, the double network hydrogel may have an increased viscosity relative to the first and/or second composition (e.g., a second viscosity). For example, in some embodiments, the viscosity of double network hydrogel as measured at 25 degrees C. is greater than or equal to 500 mPa s, greater than or equal to 1,000 mPa s, greater than or equal to 5,000 mPa s, greater than or equal to 10,000 mPa s, greater than or equal to 20,000 mPa s, greater than or equal to 50,000 mPa s, greater than or equal to 100,000 mPa s, greater than or equal to 500,000 mPa s, greater than or equal to 1,000,000 mPa s, or greater than or equal to 10,000,000 mPa s. In some embodiments, the viscosity of the double network hydrogel as measured at 25 degrees C. is less than or equal to 100,000,000 mPa s, less than or equal to 10,000,000 mPa s, less than or equal to 1,000,000 mPa s, less than or equal to 500,000 mPa s, less than or equal to 100,000 mPa s, less than or equal to 50,000 mPa s, less than or equal to 20,000 mPa s, less than or equal to 10,000 mPa s, less than or equal to 5,000 mPa s, or less than or equal to 1,000 mPa s. Combinations of the foregoing ranges is possible (e.g., greater than or equal to 500 mPa s and less than or equal to 100,000 mPa s). Other ranges are also possible. In some embodiments, the double hydrogel network may be a viscoelastic solid, and may exhibit a storage and/or loss moduli as described elsewhere herein. In some embodiments, the double hydrogel network may be a solid.

Categories of compositions for use in the invention(s) (e.g., as the first and/or second composition) can include biocompatible polymers with non-degradable backbones, such as linear anionic and branched, multi-arm polyethylene glycols (PEGs), such as a 4-arm species, but other species such as 3-arm, 8-arm, etc. can be used. In some embodiments, compositions may comprise an ionically crosslinkable polymer and/or a covalently crosslinkable polymer, as described elsewhere herein.

In some embodiments, the first and second composition may be introduced and/or interact in vivo. In some embodiments, the first composition may comprise a crosslinker solution (e.g., a solution containing a crosslinker). In some embodiments, the first composition may comprise a hydrogel precursor. In some embodiments, the second composition may comprise a crosslinker solution. In some embodiments, the second composition may comprise a hydrogel precursor.

In some embodiments, ingesting a first composition comprising a crosslinker followed by a second composition comprising a hydrogel precursor may result in a double network hydrogel. In some embodiments, ingesting a first composition comprising a hydrogel precursor followed by a second composition comprising a crosslinker may result in a double network hydrogel. In some embodiments, an average maximum dimension of the double network hydrogel may be affected by the order in which the first and second compositions are ingested. In some embodiments, a length, width, and height of the double network hydrogel, e.g., formed in situ, may independently be greater than 0.1 cm, greater than or equal to 1 cm, greater than or equal to 3 cm, greater than or equal to 5 cm, or greater than or equal to 8 cm. In some embodiments, the length, width, and height of the double network hydrogel, e.g., formed in situ, may independently be less than or equal to 10 cm, less than or equal to 8 cm, less than or equal to 5 cm, less than or equal to 3 cm, or less than or equal to 1 cm. Combinations of the foregoing ranges are possible. Other ranges are also possible.

A crosslinker solution, in some embodiments, may comprise a crosslinking reagent that may facilitate the crosslinking of polymer chains. In some embodiments, the crosslinking reagent may comprise an ion, for example, a metallic cation to facilitate an ionically crosslinked polymer network. In some cases, the crosslinking reagent may be a compound that reacts and forms covalent bonds between polymers, e.g., forming a covalently crosslinker polymer network. Those of ordinary skill in the art understand and are able to select appropriate crosslinking agents for use in pharmaceutically acceptable compositions given the benefit of the present disclosure. In some embodiments, the crosslinker solution may comprise metallic ions, such as Ca², Na⁺, Al³⁺, Zn²⁺, and/or Mg²⁺. In some embodiments, the crosslinker solution may comprise calcium chloride. In some embodiments, the crosslinker component may comprise organic compounds, including dimercaptosuccinic acid and/or polyethylene glycol-dithiol (PEG-dithiol). Other crosslinking reagents are also possible. Multiple crosslinking reagents may be present in the crosslinker solution, for example, to crosslink two different polymer networks and form a double network hydrogel. For instance, in some embodiments, the crosslinking solution may comprise calcium chloride and dimercaptosuccinic acid. In some embodiments, the crosslinking solution may comprise calcium chloride and polyethylene glycol-dithiol. In some such embodiments, at least two crosslinkers are present in the first and/or second composition. In accordance with some embodiments, the crosslinking reagents may be compatible and/or pharmaceutically acceptable.

A hydrogel precursor, according to some embodiments, may comprise any of a variety of polymers (e.g., or monomers, oligomers, or prepolymers). In some such embodiments, the polymers may be crosslinked to form a polymer network after the hydrogel precursor is introduced to the crosslinker solution. In some embodiments, the polymers may comprise a polyanionic polymer. In some such cases, the polyanionic polymers comprise carboxylic acid functional groups. In some embodiments, the polymers may comprise a polyethylene glycol. According to some embodiments, the polymers contained in the hydrogel precursor may comprise a PEG-maleimide having at least 4 legs. For example, in some cases, the PEG-maleimide may have 4 arms, 6 arms, and/or 8 arms. The multiplicity of the arms of the PEG-maleimide may facilitate more thorough crosslinking of the polymer when forming a polymer network comprising the PEG-maleimide. In some cases, the polymers contained in the hydrogel precursor may comprise alginate. Other polymers are also possible. Multiple polymers may be present in the hydrogel precursor, for example, to facilitate the crosslinking of the at least two different polymers into polymer networks. In some such embodiments, the hydrogel precursor comprises alginate and a multi-arm PEG (e.g., 4-arm PEG maleimide). In accordance with some embodiments, the hydrogel precursors may be compatible and/or pharmaceutically acceptable. In some such embodiments, hydrogel precursors may be suitable for oral administration to a subject.

According to some embodiments, the crosslinker solution may comprise multiple crosslinking agents and the hydrogel precursor may comprise multiple polymers (e.g., or monomers, oligomers, and/or prepolymers). According to some embodiments, the crosslinker solution may comprise two crosslinking agents and the hydrogel precursor may comprise two polymers. In some embodiments, each of the multiple crosslinking agents may be appropriately selected in order to crosslinker at least one of the polymers present in the hydrogel precursor. Accordingly, upon interaction of the crosslinker solution and the hydrogel precursor (e.g., the first and second compositions), each polymer of the hydrogel network and its corresponding crosslinking agent may react to form polymer networks. In some such embodiments, wherein two crosslinking agents and two polymers are present in the crosslinker solution and hydrogel precursor, respectively, two polymer networks may be formed, e.g., to form interpenetrating polymer networks, as described elsewhere herein. In some embodiments, the interpenetrating polymer networks forms a double network hydrogel.

In some embodiments, the first and/or second composition (e.g., the crosslinker solution and/or the hydrogel precursor) may further comprise a therapeutic agent (e.g., a pharmaceutically acceptable and/or active component, which may or may not be suitable for oral administration to a subject). In some cases, the therapeutic agent being present in the first and/or second composition may result in the therapeutic agent being present in a double network hydrogel formed therefrom. For example, rapid crosslinking and/or thickening of the first and second compositions may form a solid (e.g., hydrogel, semi-solid, etc.) that encapsulates at least a portion of the therapeutic agent that was present in the first and/or second composition. In some cases, a therapeutic agent comprises a chemical compound, a bacteria, a peptide, and/or other biologically active compound. Those of ordinary skill in the art would be able to select appropriate therapeutic agent based on the desired application and/or treatment. Examples of suitable therapeutic agents are described elsewhere herein.

According to some embodiments, the first and/or second composition (e.g., the crosslinker solution and/or the hydrogel precursor) may further comprise an excipient. In some cases, the excipient being present in the first and/or second composition may result in the excipient being present in a double network hydrogel formed therefrom. Those of ordinary skill in the art will understand the meaning of excipient, generally referring to pharmacologically inactive substances. In some cases, the excipient may stabilize the pharmaceutically acceptable and active component. For example, in some embodiments, the excipient may comprise calcium carbonate, which may act as a buffering compound in the interior of a resulting double network hydrogel, e.g., when the double network hydrogel is present in acidic media such as gastric fluid. Buffering capacity from an excipient may minimize and/or present the pharmaceutically acceptable and/or active components from interacting with relatively harsh environments that may breakdown the pharmaceutically acceptable and/or active components. Other types of excipients are also possible, and those of ordinary skill in the art will be able to select them based on the therapeutic agent and/or the desired application.

Double network hydrogels, in accordance with some embodiments, comprise two interpenetrating hydrogel networks. For example, in some embodiments, a first hydrogel network may comprise alginate and a second hydrogel network may comprise PEG. Double network hydrogels, in accordance with some embodiments and as described elsewhere herein, may have mechanical properties that make them suitable for applications wherein mechanical stresses are present. For instance, in some cases, the double network hydrogels disclosed herein may be forming in the stomach or other region of the gastrointestinal tract, and due to the mechanical properties of the double network hydrogels, the hydrogels may be mechanically stable and/or maintain a physical separation of cargo contained within the hydrogel from an environment outside of the hydrogel (e.g., in the stomach and/or other region of the GI tract).

The following is a non-limiting list of and example of specific compositions that can be used in the invention: dithiol-containing drugs or molecules, including FDA-approved and/or those in clinical trials, e.g., 2,3-dimercaprol, bucillamine, 2,3-dimercapto-1-propanesulfonic acid; dithiol-containing polymers that contain non-degradable backbones: e.g., dithiol-containing poly(acrylamide), poly(HPMA), poly(HEMA); these polymers may also be branched (e.g., 3-, 4-, or 8-arm); of various molecular weights (200-100 kDa); polyionic polymers that undergo rapid, ionic crosslinking: e.g., gellan gum and/or karaya gum; thermosensitive polymers such as poly(NIPAM); maleimide-containing polymers contain non-degradable backbones: e.g., maleimide-containing poly(acrylamide), poly(HPMA), poly(HEMA); these polymers may befit from being branched and of various molecular weights (10-100 kDa); other forms of calcium for crosslinking may include: CaCO₃ and may be formulated into different forms (tablet, powder, nano- or micro-particles).

Those of ordinary skill in the art can utilize understood biological and chemical principles to select these and other species as compositions for use in the invention(s). Example materials from which the compositions may be selected to form hydrogels (e.g., double network hydrogels) include those described in US patent application number 2022/0193240, which is herein incorporated by reference in its entirety. Where the invention is to be used in vivo in the GI tract, any crosslinking chemistry may be selected to occur in timescales compatible with the short residence times of liquids in the stomach (half life of approximately 30 min). Therefore, reference can be made to rapid crosslinking as reported in the academic and patent literature to which those of ordinary skill in the art have ready access. Generally, ionic crosslinking occurs rapidly (e.g., mixing of alginate with divalent cations induces nearly rapid crosslinking), so the limiting factor is the covalent (or second) network. In some embodiments, one or both of the polymer networks that form when the first and second compositions are mixed may crosslink to form a polymer network in less than or equal to 30 minutes, less than or equal to 25 minutes, less than or equal to 20 minutes, less than or equal to 15 minutes, less than or equal to 10 minutes, or less than or equal to 5 minutes. As mentioned above, in accordance with some embodiments, the ionic polymer network may crosslink relatively quicker than the covalent polymer network, and thus may crosslink to form a first polymer network in which the covalent polymer network precursors (e.g., polymer and crosslinker) may thereafter crosslink and/or interpenetrate.

In addition, a simple screening test or set of screening tests can be carried out in a typical laboratory environment to help select species that are appropriate for use in the invention(s). This can involve one or more accepted simulated GI environments, or a similar screening test. In one such test, a first composition (e.g., comprising a crosslinker or a hydrogel precursor) and a second composition (e.g., comprising a crosslinker or a hydrogel precursor) may be added to a simulated GI environment. Such a test provides a screen for the capacity of polymer networks to undergo rapid crosslinking and therefore is a screen for rapid crosslinking chemistries. Another such test such text involves drip casting a hydrogel precursor into crosslinker for short time periods (10-20 min) and then assessment of mechanical properties of the resultant, thickened product (raised in viscosity) by compression compared to single-network (alginate-only hydrogels), which may facilitate understanding the mechanical properties and/or the robustness of the resulting solids. These tests may also be performed in simulated gastric fluid (SGF) and real porcine gastric fluid (rGF).

Composition, articles, and methods of the invention can be used to deliver a therapeutic agent, active substance, or active agent to a subject. In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8^{th} edition (Sep. 21, 2000); Physician's Desk Reference (Thomson Publishing), and/or The Merck Manual of Diagnosis and Therapy, 17^{th} ed. (1999), or the 18th ed (2006) following its publication, Mark H. Beers and Robert Berkow (eds.). Merck Publishing Group, or, in the case of animals, The Merck Veterinary Manual, 9^{th} ed., Kahn, C. A. (ed.), Merck Publishing Group, 2005; and “Approved Drug Products with Therapeutic Equivalence and Evaluations.” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In certain embodiments, the artemisinin derivative is artesunate.

In some embodiments, the solid (e.g., semi-solid hydrogel, double network hydrogel, etc.) that results from the mixing of the first and second composition may have any of a variety of parameters. In some embodiments, the solid is biocompatible and/or pharmaceutically acceptable. In some embodiments, the solid comprises a double network hydrogel that is biocompatible and/or pharmaceutically acceptable. In some cases, the solid may have a toughness that may allow the solid to remain at a location internal to a subject and retain its composition, e.g., without being mechanically broken down.

In some embodiments, the solid (e.g., a double network hydrogel) may have a capacity to be mechanically deformed without breaking, physically deteriorating, or otherwise degrading after a being compressed a number of cycles. This may be advantageous for maintaining cargo within the solid when being mechanically compressed within the stomach, in some embodiments. In some embodiments, the solid may maintain its integrity (e.g., may retain a shape and/or may not break into multiple components) after being compressed at least 2 times, at least 3 times, at least 5 times, at least 10 times, at least 20 times, at least 50 times, at least 100 times, or at least 500 times. In some embodiments, the solid may maintain its integrity (e.g., may retain a shape and/or may not break into multiple components) after being compressed no more than 1.000 times, no more than 500 times, no more than 100 times, no more than 50 times, no more than 20 times, no more than 10 times, no more than 5 times, or no more than 3 times. Combinations of the foregoing ranges are possible. Other ranges are also possible. In some embodiments the solid may maintain its integrity after withstanding a compressive load of at least 10 kPa, at least 15 kPa, or at least 20 kPa. In some cases, the solid maintains its integrity after withstanding a compressive load of no more than 25 kPa, no more than 20 kPa, or no more than 15 kPa. Combinations of the foregoing ranges are possible. Other ranges are also possible. In some embodiments, the solid maintains elasticity after being deformed at least 70%, at least 80%, at least 90%, or at least 95%. In some embodiments, the solid maintains its elasticity after being deformed no more than 98%, no more than 95%, no more than 90%, or no more than 80%.

In some embodiments, the solid (e.g., semi-solid hydrogel, double network hydrogel, etc.) that results from the mixing of the first and second composition may have any of a variety of storage moduli. In some embodiments, the storage modulus of the solid may be greater than or equal to 100 Pa, greater than or equal to 300 Pa, greater than or equal to 500 Pa, greater than or equal to 800 Pa, or greater than or equal to 1000 Pa. In some embodiments, the storage modulus of the solid may be less than or equal to 1200 Pa, less than or equal to 1000 Pa, less than or equal to 800 Pa, less than or equal to 500 Pa, or less than or equal to 300 Pa. Combinations of the foregoing ranges are possible. Other ranges are also possible.

In some embodiments, the solid (e.g., semi-solid hydrogel, double network hydrogel, etc.) that results from the mixing of the first and second composition may have any of a variety of loss moduli. In some embodiments, the loss modulus of the solid may be greater than or equal to 20 Pa, greater than or equal to 50 Pa, greater than or equal to 75 Pa, greater than or equal to 100 Pa, or greater than or equal to 120 Pa. In some embodiments, the storage modulus of the solid may be less than or equal to 140 Pa, less than or equal to 120 Pa, less than or equal to 100 Pa, less than or equal to 75 Pa, or less than or equal to 50 Pa. Combinations of the foregoing ranges are possible. Other ranges are also possible.

According to some embodiments described above, the first and second composition may be ingested. In other embodiments, the first and second compositions may be applied to a location internal to a subject in other manners, e.g., by using a double barrel syringe. In some such embodiments, the use of a double barrel syringe or other application method other than ingestion may facilitate the use of the compositions described herein at various locations internal to the body and/or for various applications. In some embodiments, the rapid crosslinking of the solid (e.g., double network hydrogel) may facilitate the application of a layer of the solid to a portion of the body (e.g., an open wound, a tissue, etc.), e.g., that is internal to the body.

In some embodiments, the location internal the body may comprise a location outside of the GI tract. In some such cases, the compositions described herein may need to be altered to remain biocompatible and/or pharmaceutically acceptable. For example, in some cases as described elsewhere herein, the amount of a component may dictate its biocompatibility and/or pharmaceutical acceptability. In some embodiments, for example, a first amount of calcium suitable for oral delivery may need to be lessened to a second amount of calcium to be suitable for another type of delivery. Those of ordinary skill in the art will be able to appropriately select amounts of each precursor (e.g., hydrogel precursor and/or crosslinker). In some embodiments, the location internal comprises the rectum, the vagina, and/or the urethra. In some embodiments, the location internal to the subject comprises a subcutaneous area, e.g., accessed by injection.

The following examples are intended to illustrate certain embodiments of the present invention, but does not exemplify the full scope of the invention.

### Example 1

In this example, an in situ-forming, double network hydrogel for oral delivery of a diverse set of drugs is described. The formulation comprises two “drinks” that once mixed within the stomach, form tough materials that are able to withstand the forces of the gastrointestinal tract. Therefore, this is a liquid-to-solid-transitioning system. In combination with excipients, these hydrogels have the capacity to protect encapsulated drugs against the harsh environment of the gastrointestinal tract.

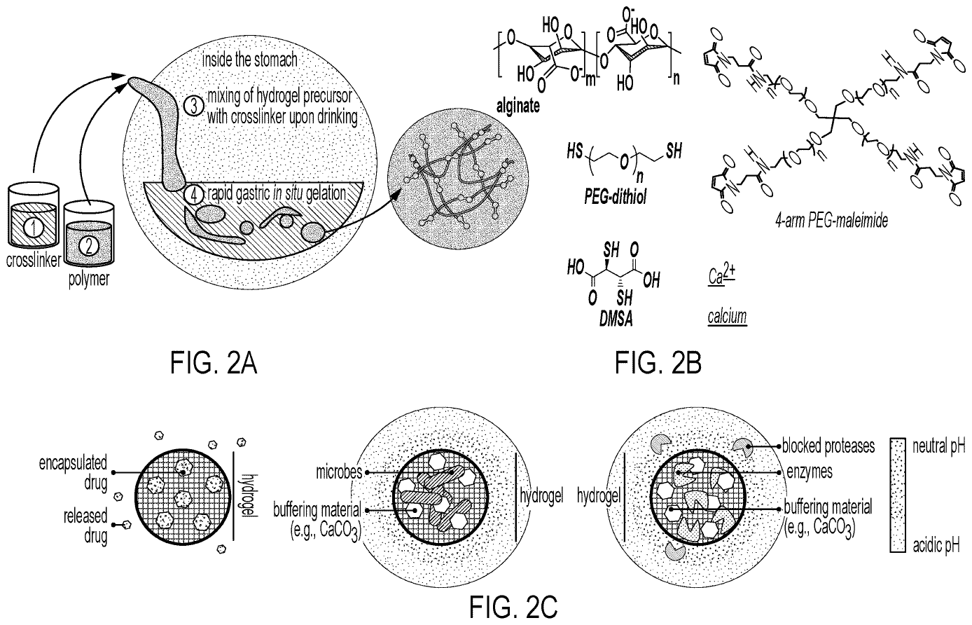

A new strategy to facilitate a drinkable, liquid in situ-forming and tough excipient, which can be a hydrogel, is described. In one set of embodiments, this can comprise both ionic (calcium/alginate) and covalent (poly(ethylene glycol) (PEG)) polymer networks for enhanced toughness. These hydrogels undergo gelation after the liquid polymer solution containing alginate and functionalized PEG contacts the crosslinker solution (FIGS. 2A-2B). Extensive characterization of LIFT (liquid in situ¬-forming and tough) hydrogels after ex vivo formation in real gastric fluid and in vivo formation in rodent and large porcine models is performed, and the capacity of the LIFT hydrogels to form solids in situ is demonstrated and allows these materials to act as a depot for controlled release of small molecules. Moreover, LIFT hydrogels can retain CaCO₃ as an excipient and preserve the activity of orally delivered enzymes in rodent and porcine models (FIG. 2C).

First, the patient imbibes a crosslinker drink comprising calcium ions and dithiol-containing molecules. Next, the patient ingests the hydrogel precursor comprising alginate and a 4-arm poly(ethylene glycol)-maleimide. Once the second solution comes into contact with the first, it forms a solid hydrogel that is mechanically tough, for example, due to the presence of the double network of hydrogels comprising ionic and covalent polymer networks. The solid hydrogel is then mechanically characterized to demonstrate their capacity to deliver and/or protect small molecules, probiotics, and/or enzymes.

To bridge the advantages of solid and liquid dosage forms, a robust crosslinking chemistry capable of rendering tough, double-network hydrogels within the stomach is described. These LIFT (liquid in situ¬-forming and tough) hydrogels are formed through oral ingestion of a crosslinker solution of calcium and dithiol crosslinkers, followed by the ingestion of a polymer solution of alginate and 4-arm poly(ethylene glycol)-maleimide. It is shown that LIFT hydrogels are able to robustly form in the presence of complicated gastric fluid and in vivo in rat and porcine stomachs, and are mechanically tough. LIFT hydrogels are retained within the porcine stomach for up to 24 h and are biocompatible. These hydrogels exhibited comparable total released drug as unencapsulated drug but with reduced drug plasma concentrations. Co-encapsulation of lactase (e.g., as a model biologic drug) and calcium carbonate (e.g., as an excipient) mitigated gastric-mediated deactivation of the LIFT-encapsulated enzyme in rat and porcine models. LIFT hydrogels present a biocompatible and robust means of tough, double-network hydrogel formation in situ in the gastric cavity, and may expand medication access for patients with difficulty swallowing.

### Example 1

### Results

Due to the relatively short residence times (<30 min) of liquids in the stomach and the complexity of gastric fluid, of crosslinking chemistries that rapidly and robustly crosslink two, interpenetrating polymer networks was developed. Alginate is a well-studied, biocompatible polymer derived from algae with generally recognized as safe (GRAS) status; alginate polymers contain blocks of consecutive and/or alternating β-D-mannuronate and α-L-guluronate residues, the latter of which undergoes nearly instant crosslinking in the presence of calcium. Accordingly, alginate was used as the first polymer network. PEG was utilized for the second network due to the established safety profiles of ingested PEGs. Three, conventionally used chemistries were initially considered during development: NHS ester/amine, DBCO/azide, and maleimide/thiol. Due to the evolution of an uncharacterized and potentially toxic NHS leaving group during NHS ester/amine reaction and the slow kinetics (>1 h) of commercially available DBCO-/azide-functionalized PEGs, a PEG network crosslinked by maleimide/thiol reaction was used. Advantages of this chemistry include its rapid reaction kinetics, mild reaction conditions, and biocompatibility. To mitigate the risk of crosslinker toxicity, small molecular weight, FDA-approved or GRAS dithiol-containing molecules for rapid diffusion and crosslinking of maleimide-functionalized PEG were desirable. Dimercaptosuccinic acid (DMSA) was selected as a small molecule dithiol crosslinker due to its FDA approval status, extensive use history, and well-characterized safety profile in children and adults. A dithiol-terminated linear PEG (MW=1000 Da) was also selected for evaluation. Therefore, the final concept comprises (1) ingestion of a crosslinker solution comprising calcium chloride and DMSA or PEG-dithiol (e.g., CaCO₃ and DMSA, CaCO₃ of PEG-dithiol), followed by (2) ingestion of a liquid polymer solution comprising alginate and 4-arm PEG-maleimide. Upon (3) mixing in the stomach, the liquid polymer solution undergoes crosslinking of both polymer networks and gelation to form (4) LIFT hydrogels (FIGS. 2A-2B).

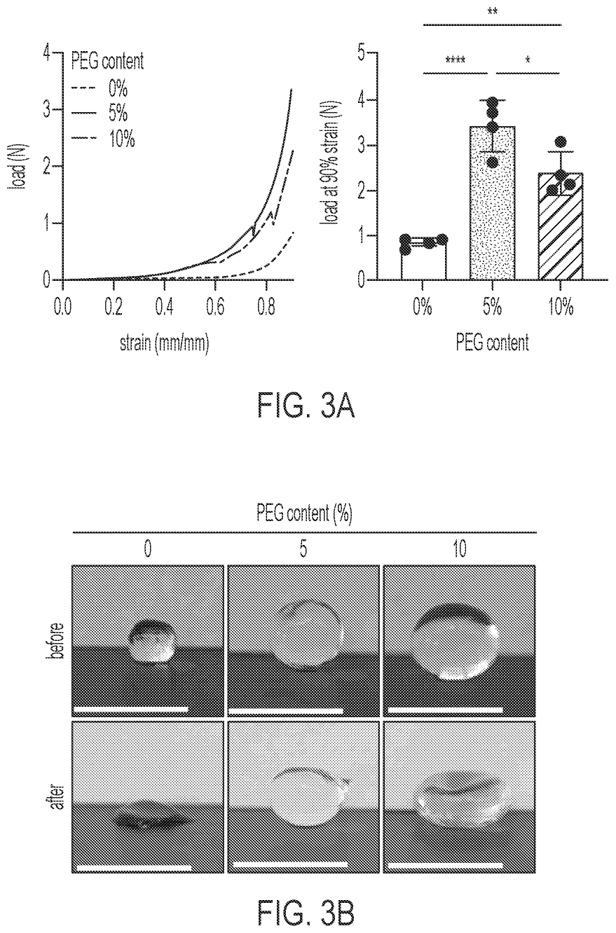

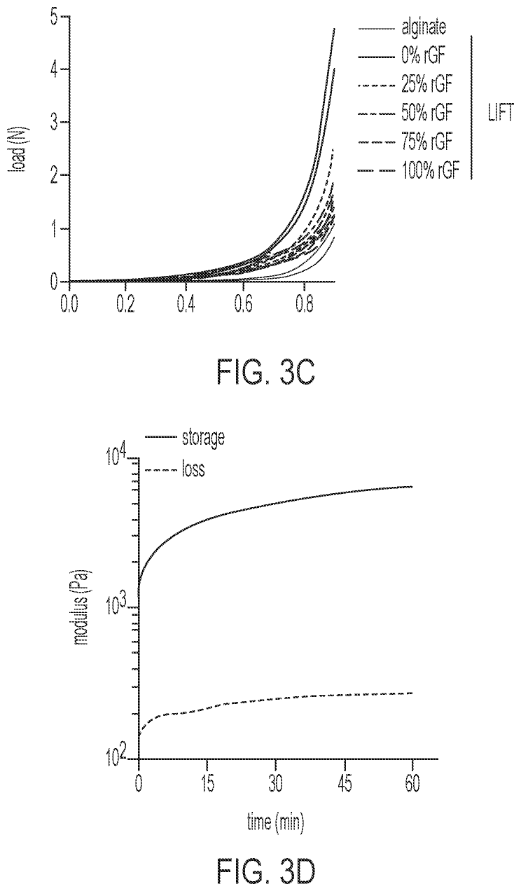

LIFT hydrogels were tested to see if they were capable of forming (e.g., crosslinking) under short (e.g., less than or equal to 20 min, 15 min, 10 min, 5 min, or so forth as described elsewhere herein) time durations relevant to gastric residence of ingested liquids. To emulate in vivo formation conditions, solutions containing a 0.5 wt % alginate with 0, 5, or 10% 4-arm PEG-maleimide were drop cast into a crosslinker solution (200 mM CaCl₂/10 mM PEG-dithiol or DMSA) and then incubated for 10-20 min at 37° C. The resulting hydrogels were mechanically characterized by compression testing. Notably, alginate hydrogels containing a crosslinked PEG network sustained significantly greater loads compared to alginate-only hydrogels (FIG. 3A). After 90% strain, LIFT hydrogels remained mostly spherical, whereas alginate-only hydrogels remained permanently deformed (flattened) (FIG. 3B). LIFT hydrogels were further mechanically characterized by cyclic compression testing. While LIFT hydrogels could sustain at least 5 cycles of 90% strain, alginate-only hydrogels remained permanently deformed after 1 cycle and were unable to sustain subsequent strains. Due to the greater mechanical performance and easier manipulation of 0.5% alginate/5% PEG-containing hydrogels compared to 10% PEG-containing hydrogels, this composition was further characterized. This observation may be due to greater dissolution and mixing of the 5% w/v (e.g., percent weight solute by volume solvent) PEG with alginate. To test the capacity of LIFT hydrogels to form in vivo, hydrogels were formed in fresh porcine gastric fluid at various dilutions in water. As a control, hydrogels were compared to LIFT or alginate-only hydrogels formed in the absence of gastric fluid. While gastric fluid attenuated the mechanical properties of LIFT hydrogels, these hydrogels were still mechanically tougher than alginate-only hydrogels formed under ideal conditions (e.g., in water; FIG. 3C). LIFT hydrogel components were also tested for cytotoxicity in cultured human colon epithelial (Caco-2, HT-29), mouse liver (Hepa1-6), and monkey kidney (CV-1) cells. After 24 h of continuous incubation at relevant concentrations, no major causes of cytotoxicity were observed. Collectively, these data demonstrate that LIFT hydrogels can form rapidly even in gastric fluid, the resulting hydrogels are mechanically tough, and that both DMSA and PEG-dithiol crosslinkers are capable of crosslinking the covalent PEG network.

The kinetics of LIFT hydrogel formation were further studied by rheometry. Notably, the sharpest increase in modulus occurred during the first 10-15 min (FIG. 3D), further supporting the feasibility of gastric crosslinking at timeframes relevant to liquid retention within the stomach (time of 50% emptying: 15-30 min). LIFT hydrogels were then studied for their capacity to encapsulate therapeutic cargos of different length scales, using 155-kDa fluorescent dextran as a model macromolecular enzyme, and 20- or 100-nm fluorescent polystyrene nanoparticles as model control-release nanoparticles. The ability to co-encapsulate and detain cargos and excipients may facilitate protection of cargo function in the harsh gastrointestinal environment. LIFT or alginate-only hydrogels encapsulating these model cargoes were immersed in simulated gastric fluid (SGF, pH 1.77) or simulated intestinal fluid (SIF, pH 6.8), which were sampled at various timepoints. Neither hydrogel were able to detain dextran in either media (>75% release); however, LIFT hydrogels exhibited less nanoparticle release in SIF (<1-6%) compared to alginate-only hydrogels after 24 h (70-77%). This suggests the increased pore sizes and release of cargo from alginate hydrogels in alkaline environments. Therefore, LIFT hydrogels may be capable of retaining therapeutic cargoes at a variety of time length scales due to greater stability at various pH ranges and/or smaller pore sizes.

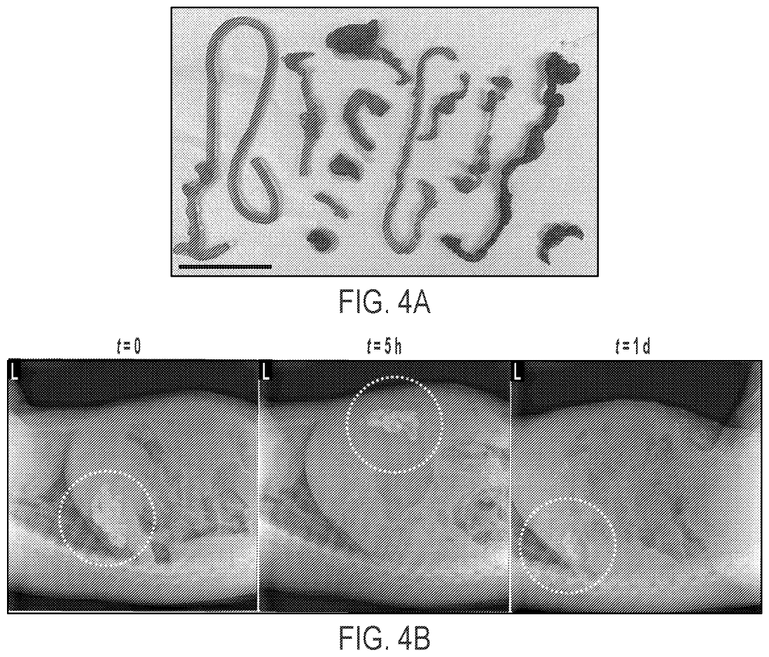

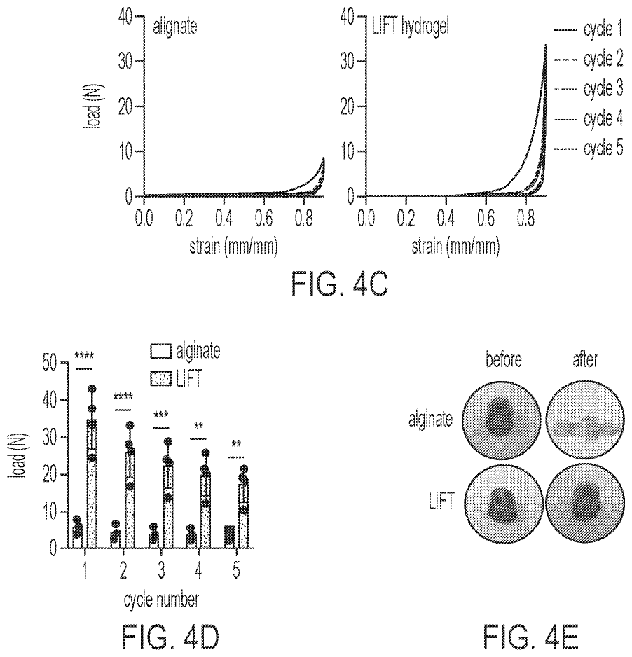

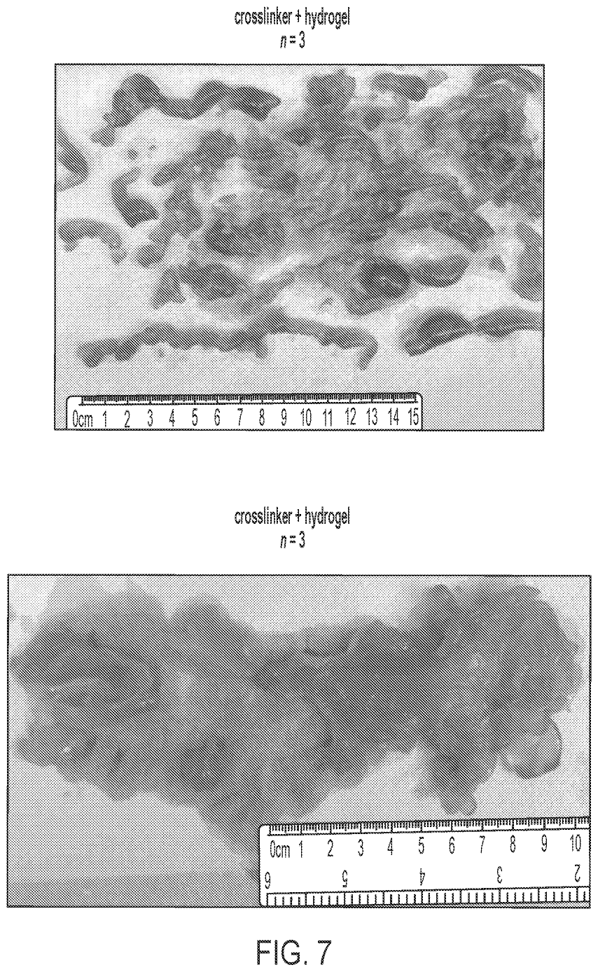

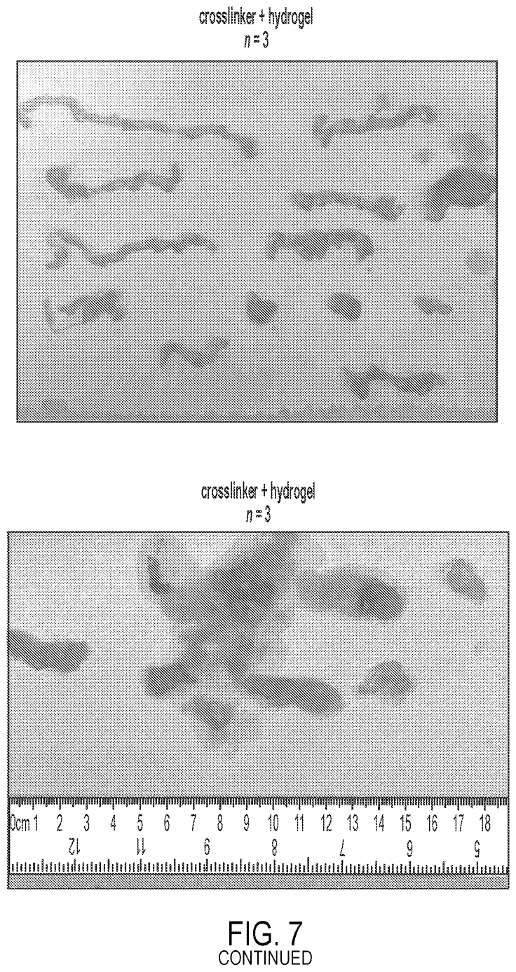

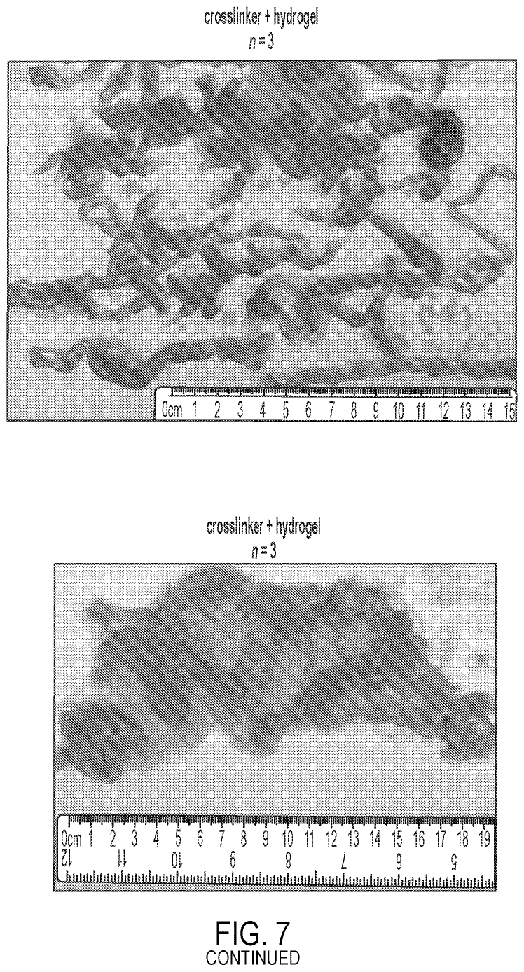

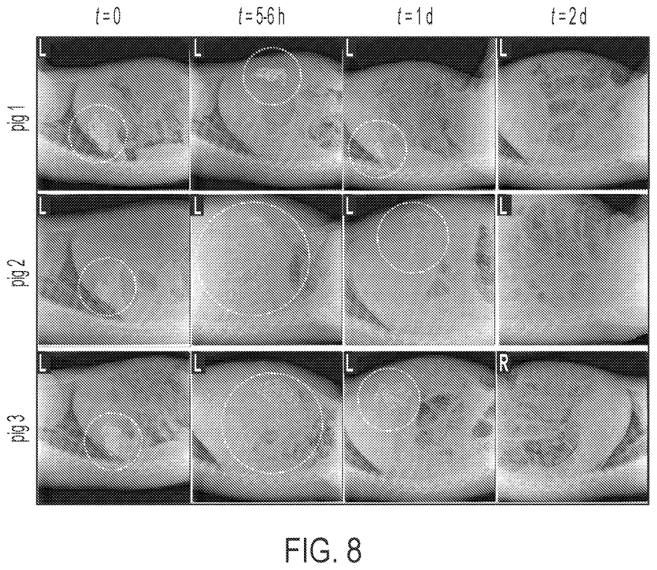

LIFT hydrogels were then tested and characterized for formation, kinetics, and safety in vivo. Porcine models were tested due to the similarity in size of the gastrointestinal tract to that of humans. First, the administration order of crosslinker (200 mM CaCl₂/10 mM DMSA or PEG-dithiol, e.g., a first composition) and hydrogel precursor (0.5% alginate/5% 4-arm PEG-maleimide, e.g., a second composition) was varied. Pigs were administered solutions via endoscope, and hydrogel structures were retrieved and studied 5-8 h afterwards. Hydrogels formed within the stomach cavity regardless of administration order. Administration of crosslinker solution first and then hydrogel precursor resulted in the reproducible formation of noodle-like hydrogels within the stomach; conversely, administration of hydrogel precursor first and then crosslinker resulted in larger bulk hydrogels (FIG. 4A, FIGS. 7-8). Accordingly, LIFT hydrogels were formed in vivo by first administration of the crosslinker solution followed by the hydrogel precursor due to greater consistency in hydrogel architectures. LIFT hydrogels were then studied for their transit time in vivo through X-ray imaging of hydrogels containing 20% barium sulfate. In general, LIFT hydrogels remained within the stomach up to 24 h after formation (FIG. 4B, FIG. 8). No major changes in serum alkaline phosphatase, aspartate aminotransferase, alanine aminotransferase, blood urea nitrogen, or creatinine were observed up to 48 h after administration (Table 1). These data support that LIFT hydrogels and their components are safely cleared, do not cause obstructions, and do not cause toxicity.

TABLE 1
Porcine blood chemistry after LIFT hydrogel.

| Time | ALP (U/L) | AST (U/L) | ALT (U/L) | BUN (mg/dL) | creatinine (mg/dL) |
|---|---|---|---|---|---|
| baseline | 131.0 ± 25.7 | 18.7 ± 4.0 | 41.7 ± 6.8 | 8.7 ± 0.6 | 1.1 ± 0.1 |
| 24 h | 132.3 ± 22.5 | 22.3 ± 17.2 | 41.7 ± 6.1 | 12.7 ± 4.5 | 1.2 ± 0.2 |
| 48 h | 115.0 ± 16.5 | 22.3 ± 5.5 | 38.7 ± 5.9 | 9.0 ± 1.7 | 1.1 ± 0.2 |

ALP, alkaline phosphatase; AST, aspartate aminotransferase; ALT, alanine transaminase; BUN, blood urea nitrogen.
Baseline: sample was collected before LIFT hydrogel administration. An n = 3 pigs were tested.

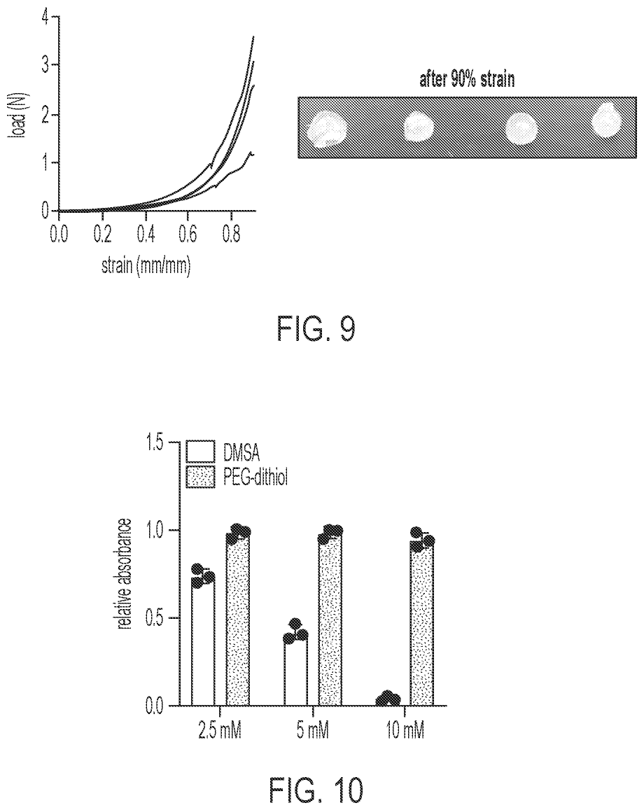

After formation in the gastric cavity, LIFT hydrogels were characterized for their mechanical properties by cyclic compression testing. Similar to in vitro experiments, LIFT hydrogels were tougher and able to sustain at least 5 cyclic 90% strains, whereas alginate-only hydrogels remained flattened after 1 cycle (FIGS. 4C-4E, FIG. 9). These findings highlight the capacity of the LIFT hydrogels to robustly form in the stomach after oral administration in a human-scale gastrointestinal tract.

Gastrointestinal mucus is abundant with cysteines, which may react with thiol and maleimide groups present within LIFT. This may impact hydrogel yield or cause hydrogel adhesion to gastric tissue, as has been leveraged in other systems. To test the impact of tissue on hydrogel yield, crosslinker was applied directly to tissue or a plastic plate as a control. A modified Franz diffusion device was utilized to create individual “wells” on top of stomach tissue for this experiment. Hydrogel yield, defined by mass, did not significantly differ between formation in a gastric tissue environment or normal plastic plate, regardless of whether DMSA or PEG-dithiol was used. Moreover, formation in a gastric tissue environment did not appear to negatively impact hydrogel mechanical properties. Thus, side reactions with tissue do not seem to occur at a scale that significantly impacts hydrogel formation. LIFT adhesion to tissue was tested using a tilt test. After incubation, tilting, and washing of wells, LIFT adhesion to gastric tissue was not observed. This is in agreement with the in vivo porcine experiments, in which hydrogel adhesion after endoscopic delivery was not observed, and may be due to saturation of tissue thiols and/or PEG-maleimide groups with crosslinker thiols.

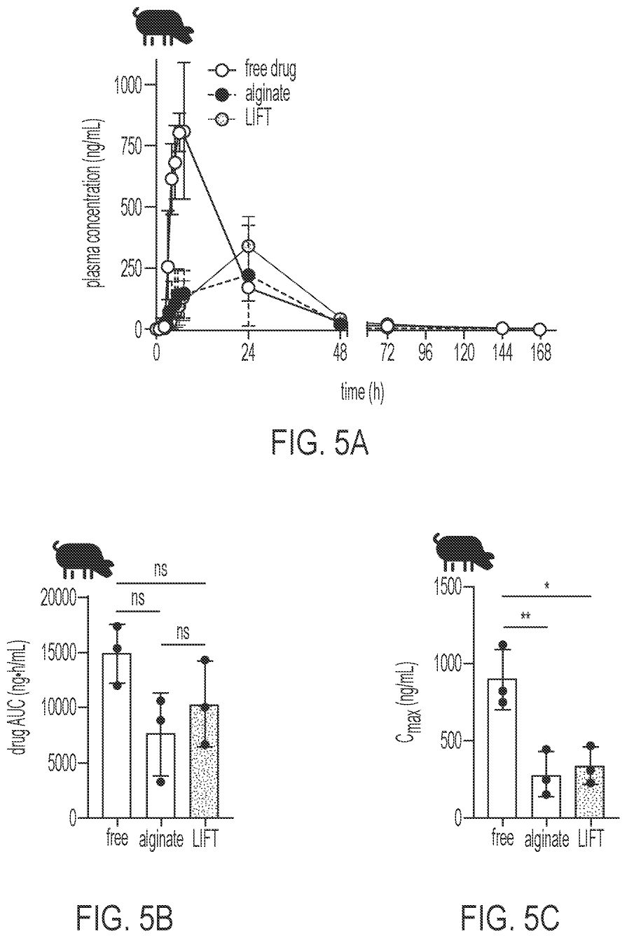

Having established that LIFT hydrogels can form in vivo, their capacity to encapsulate and modulate small molecules and biologic enzyme drugs with excipients was evaluated. Lumefantrine was selected as a model small molecule drug because of its poor solubility in water and, hence, would form a drug suspension that would be encapsulatable within the hydrogel after formation. Hydrogels were administered as above (e.g., crosslinker solution followed by hydrogel) in pigs using 200 mM CaCl₂)/10 mM DMSA as the crosslinker solution; lumefantrine was suspended in 0.5% alginate/5% 4-arm PEG-maleimide LIFT polymer solution. Lumefantrine powder loaded in gelatin pills was used as a free drug control, and all pigs were dosed with 960 mg lumefantrine. Whereas free lumefantrine resulted in peak plasma concentrations at 5-7 h post-administration, hydrogel (alginate and LIFT) formulations resulted in peak plasma drug concentrations at ˜24 h (FIG. 5A). The area under the curve (AUC) of released drug from free drug, alginate, LIFT hydrogel formulations was 14,873.5±2,719.2, 7,568.4±3,780.6, and 10,337.5±3,849.7 ng·h/mL, respectively, and was not statistically different (FIG. 5B). While drug AUCs did not differ, the maximum observed drug concentration (Cₘₐₓ) was significantly higher with free drug (901.2±197.1 ng/mL) compared to alginate (283.8±147.3 ng/mL) and LIFT (338.7±112.6 ng/mL) hydrogel formulations (FIG. 5C). These data collectively support the capacity of LIFT hydrogels to deliver equivalent total doses of drug as free drug at lower plasma concentrations (e.g., over longer periods of time), which may regulate and/or reduce drug exposure and thus toxicity. This system could be compatible with water-soluble drugs encapsulated within controlled-release particles suspended within the LIFT hydrogel, and/or mixed with drug powder to form tough in situ depots.

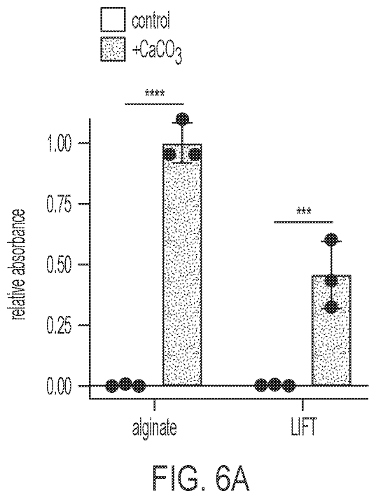

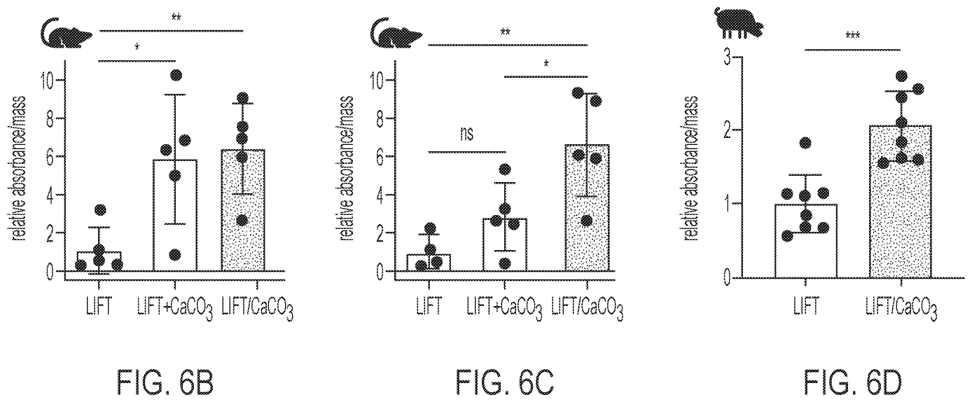

The capacity of LIFT hydrogels for oral delivery of enzymes was evaluated, which is challenging due to the acidic gastric fluid and proteases present within the gastrointestinal tract. B-galactosidase (lactase) was selected as a model enzyme therapeutic due to the need for prolonged exogenous lactase activity in the stomach to mitigate the symptoms of lactose intolerance. Indeed, lactase activity was found to be rapidly lost when incubated in SGF compared to PBS. Lactase was then encapsulated in alginate or LIFT hydrogels, along with calcium carbonate (CaCO₃) as an excipient to neutralize the acidic gastric fluid. CaCO₃ was selected because it is water-insoluble and therefore detainable within the hydrogels, and because of its GRAS status. Because the DMSA crosslinker attenuated lactase activity (FIG. 10), these LIFT hydrogels utilized the PEG-dithiol crosslinker. When incubated in SGF, only CaCO₃-containing alginate and LIFT hydrogels preserved lactase activity (FIG. 6A), underscoring the need to facilitate co-encapsulation of excipients. In addition to acidic gastric fluid, the gastrointestinal tract is rife with proteases that are capable of degrading enzymes. The capability of hydrogels of protecting cargo (e.g., encapsulated molecules) against trypsin as a model protease was tested. Only LIFT hydrogels significantly rescued lactase activity compared to free lactase or lactase encapsulated in alginate hydrogels. Therefore, in addition to co-encapsulating CaCO₃, LIFT hydrogels may exhibit additional barriers against exterior proteases due to the denser, dual polymer networks compared to alginate-only hydrogels, which may be useful when protecting cargo contained within the dual polymer networks. This combined with the increased toughness may sustain longer controlled release of compounds after ingestion.

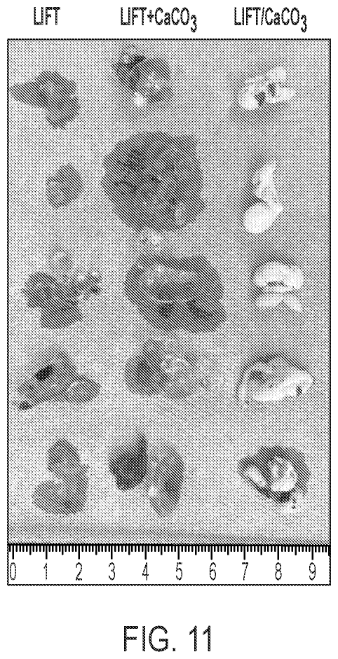

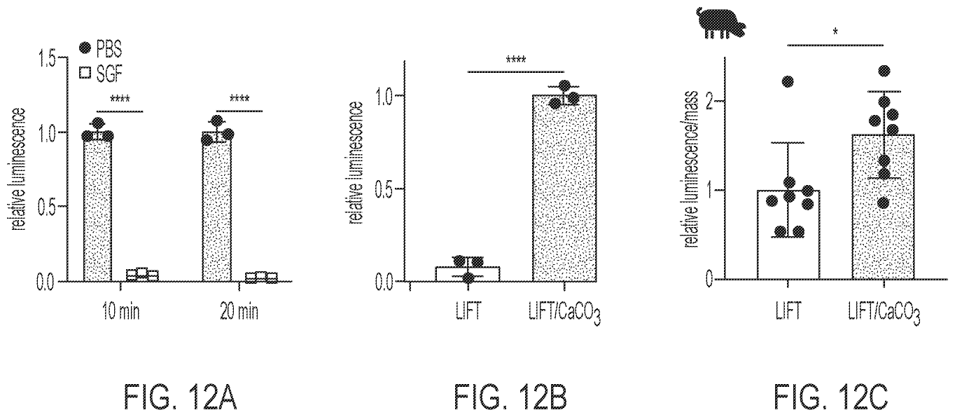

LIFT hydrogels were then tested for their ability to protect lactase activity in rat and porcine models. Analysis was focused on LIFT instead of alginate hydrogels due to their capacity to protect encapsulated lactase against exogenous proteases. Similar to studies performed in pigs, rats were first administered the crosslinker solution by oral gavage immediately followed by the hydrogel precursor containing lactase. Lactase was mixed in the hydrogel precursor with or without CaCO₃; as an additional control, CaCO₃ was suspended in the crosslinker solution. Each animal was treated with a CaCO₃ dose less than the maximum daily dose of 8-10 g/day (assuming a 75 kg human) established by manufacturers and the U.S. FDA. Therefore, these set of treatments test the effect of CaCO₃ administered separately (LIFT+CaCO₃) or co-encapsulated (LIFT/CaCO₃). Oral gavage also resulted in robust hydrogel formation in rat stomachs (FIG. 11), and hydrogels were retrieved after in vivo incubation in stomachs and assayed for lactase activity. Notably, while separate and co-encapsulated CaCO₃ significantly protected lactase activity after 1 h (FIG. 6B), only co-encapsulated CaCO₃ protected lactase after 2 h (FIG. 6C). LIFT hydrogels with co-encapsulated CaCO₃ resulted in 6-fold higher activity compared to control. The protective effect of the LIFT hydrogels co-encapsulating CaCO₃ was also observed in porcine models after a 6 h in vivo incubation (2-fold higher than control, FIG. 6D), underscoring the advantage of oral systems capable of excipient co-encapsulation even in large animal models.

To test if LIFT is broadly compatible with enzyme encapsulation, additional experiments were conducted with α-galactosidase and cellulase, two important enzyme supplements for patients with digestive enzyme deficiencies or irritable bowel syndrome. Short incubations in gastric acid significantly degraded enzyme activity. In vitro, LIFT/CaCO₃ protected both enzymes against acid-triggered inactivation; further experiments in rats also demonstrated that LIFT/CaCO₃ could protect cellulase activity after 1 h in rat stomachs.

To expand the application of LIFT, its capacity to encapsulate and protect L. lactis was examiner, which is another critical chassis for synthetic biology therapeutics. A luminescent ATP quantification assay to query bacterial viability was used. Similar to observations with EcN, L. lactis exhibited decreased viability after short exposures to SGF pH 1.77, which was mitigated when co-encapsulated with CaCO₃ within LIFT. After 6-7 h post-administration within porcine stomachs, L. lactis co-encapsulated with CaCO₃ within LIFT exhibited greater viability compared to control. The differences in response between EcN and L. lactis may be due to greater susceptibility of the latter to pH ranges relevant to gastric acid. Thus, LIFT hydrogels are capable of supporting bacterial viability and protect against acid challenge when loaded with CaCO₃ in an in vivo context. Given that acid secretion can vary 40-71 mmol/h (interquartile range) in humans and the recommended maximum recommended dose of CaCO₃ (10 g/day), these systems could potentially support the viability of bacteria in the stomach for at least 3-5 h.

### DISCUSSION

Dysphagia and difficulty swallowing pills present major obstacles to oral drug administration in older adults and pediatric patients. This is especially challenging given the increased morbidity and need for medication with advanced age: an estimated 65% patients over 65 years of age are taking at least two medications, with 37% taking more than five. Difficulty taking pills may drive patients to skip doses or modify them in ways that dangerously alter drug pharmacokinetics. In this work, a hydrogel formulation was developed, called LIFT hydrogels, capable of transitioning from liquid-to-solid upon mixing with ingested crosslinkers in the stomach, recognizing the advantages of solid formulations which confer enhanced gastric retention, protection against gastrointestinal proteases, toughness compared to liquid formulations, as well as co-encapsulation of drug with excipients. To realize LIFT hydrogels, FDA-approved or GRAS materials were utilized: alginate and 4-arm PEG-maleimide as hydrogel networks, and calcium chloride and DMSA or PEG dithiol as crosslinkers. The alginate/PEG solution remains a liquid until contact with the crosslinker solution within the stomach, facilitating a transition from a liquid to a tough hydrogel.

The gastric environment exhibits some features amenable for in situ crosslinking reactions. The stomach is temperature-controlled at 37° C., which can accelerate maleimide/thiol thioether formation; the stomach is also mechanically active and its movement could facilitate mixing of the two ingested solutions. It was demonstrated that crosslinking of both alginate and PEG networks readily occur in ex vivo porcine gastric fluid and in vivo in porcine stomachs, which underscore the robustness of the calcium- and dithiol-mediated crosslinking reaction of the alginate and 4-arm PEG-maleimide networks. While LIFT hydrogel crosslinking and mechanical properties were dependent on the proportion of gastric fluid volume, this may be diluted through greater volumes of crosslinker. The fasted stomach contains 25-35 mL of gastric fluid, which after ingestion of a 200 mL crosslinker solution is diluted to 11-15%. This proportion of gastric fluid is well within the range capable of crosslinking both networks, and the crosslinker volume is less than the volume of a typical soda can (355 mL). Notably, these reactions do not generate side products, and the hydrogels did not appear to be toxic to cultured gastrointestinal epithelial, kidney, and liver cells, nor cause clinical (e.g., constipation, inappetence) or laboratory signals in pigs up to 48 h after administration.

Gastric drug depots should be able to withstand compressive forces within the stomach to preserve depot integrity. While hydrogels with dual networks are mechanically stronger than single-network hydrogels, current strategies to formulate dual network hydrogels from orally administered liquids have not yet been described. Liquid systems have generally relied on single-network hydrogels of alginate, gellan, and karaya gum that are crosslinked by calcium. In an alternative strategy, Li et al. utilized pH-triggered unmasking of multivalent cyclodextrin to undergo gelation with multivalent adamantane in acidic conditions; however, the liberated masking group will need to be characterized for safety before application. Orally administrable tough hydrogels require radical polymerization of toxic acrylamide monomer (e.g., or other monomers) that cannot be safely performed in vivo and are dosed as a solid. Other hydrogel systems have been designed that require UV light to facilitate polymer crosslinking, utilize polyacrylamide as a polymer network, require a specific construction of hydrogel components, or are enzymatically polymerized. While these hydrogels are mechanically tough, they either require a pre-solidified dosage format or are challenging and unsafe to crosslink and gel in situ. This example facilitates liquid formulation of a tough hydrogel system. Assuming a spherical hydrogel, it is calculated that reported gastric stresses (˜13 kPa) would cause a strain of 5-10% in LIFT hydrogels, which should not permanently deform these hydrogels.

While nano- and microparticle liquid suspensions have been developed, an important feature that this system confers is the in situ gelation of macroscale structures, which are important to minimize exposure of the dosage forms to gastric fluid. Given the same volume of material, nano- and microscale dosage forms result in significantly greater surface area-to-volume ratios compared to macroscale forms. Economou et al. showed that the dissolution rate of CaCO₃ particles was size-dependent, with larger 2-4 mm particles dissolving slower than <250 μm particles in acid. Therefore, in situ gelation of macrostructures could be advantageous and facilitates protection of encapsulated therapeutics through size and geometry. Moreover, the formation of macroscale solids could prolong the gastric retention of encapsulated drugs compared to nano- and microparticulate systems liquid suspensions.

This work can alter oral small molecule pharmacokinetics and prolong the function of biological drugs within the stomach. Patients who have difficulty swallowing solids may resort to crushing their pills, which results in dramatically altered pharmacokinetics that may cause severe complications and death. Here LIFT hydrogels were shown to modify pharmacokinetics by reducing the drug plasma concentration while achieving a comparable AUC as free drug. This is significant because high drug concentrations can result in adverse side effects and impact treatment tolerability, and is applicable for drugs in which efficacy is driven by AUC and not blood concentration (e.g., tetracyclines, cytotoxics). LIFT could also control water-soluble drug release, which would require encapsulation in nanoparticles or microparticles or covalent attachment to LIFT polymers to prevent burst release. Protease- or pH-sensitive linkers could be included to further control drug release at specified gastrointestinal tissue sites. Formulation in LIFT confers additional advantages in longer transit times and reduced surface area-to-volume ratios compared to particles or drug alone that further contribute to controlled release behavior. Thus, as LIFT traverses the gastrointestinal tract, water-soluble drug is released in a form-factor that has multiple mechanisms of controlling drug release.

Given that a range of molecules are in equilibrium between the blood and gastrointestinal tract, systems that modulate these molecules within the stomach could significantly impact healthcare via a noninvasive route. Oral enzyme therapies are being developed for the treatment of hyperoxaluria and phenylketonuria, and are also used to treat patients with exocrine pancreatic insufficiency. Coupling LIFT hydrogels with these therapeutics could alter drug pharmacokinetics and prolong both their residence and function within the gastrointestinal tract in a tough form factor. LIFT could serve as a synthetic, compliant “niche” by co-encapsulating excipients (e.g., CaCO₃ as demonstrated in this work) that modulate the gastrointestinal environment and the therapeutic themselves. As LIFT hydrogels transit through the gastrointestinal tract, the porosity of the hydrogels facilitates engagement and modulation of host metabolites by the encapsulated therapeutics, or in the case of engineered bacteria, secretion of therapeutic factors. In some embodiments, drinkable and degradable crosslinkers (e.g., peptides) could be incorporated for programmed disassembly of LIFT in specified regions of the gastrointestinal tract or other regions internal to the subject. In doing so, in some embodiments, LIFT hydrogels expand the accessibility of these therapeutics to patients who otherwise have difficulty swallowing solids.

The chemistry of the LIFT hydrogels is robust, flexible, and tailorable. Here, DMSA is shown as a novel and FDA-approved small molecule crosslinker for these hydrogels, as well as a PEG-dithiol. Both crosslinkers were able to crosslink the 4-arm PEG-maleimide when mixed within the gastric cavity. The inclusion of PEG also facilitates facile covalent conjugation of drugs and other modulators using commercially available, functionalized multi-arm PEGs while still acting as a crosslinker. While maleimide-thiol reactions are rapid, inclusion of the alginate network not only augments the mechanical properties of the hydrogel, but also its immediate formation in calcium solution acts as a “template” that facilitates retention and crosslinking of the slower-forming maleimide-thiol thioether bond. PEG networks typically require highly defined maleimide:thiol ratios for efficient gelation that is challenging to implement in an oral setting; here, templating within alginate likely facilitates gradual diffusion of the dithiol crosslinkers into the hydrogel and subsequent formation of the PEG network.

By overcoming the “ship-in-a-bottle” problem, LIFT hydrogels could expand access to medications for patients who have difficulty swallowing and bridge the advantages of solid and liquid drug formulations. Through careful selection of materials, LIFT hydrogels comprise two biocompatible polymer networks that are able to crosslink in situ within the stomach, resulting in a strong hydrogel that can facilitate localization of drugs and excipients and withstand the compressive forces of the gastrointestinal tract. LIFT hydrogels are safe, and are capable of modulating small molecule release and protecting therapeutic enzymes in the stomach of large animals. In some embodiments, LIFT hydrogels and their flexible chemistries may be a useful strategy with applications in gastric drug modulation and delivery, weight loss, and protection of encapsulated biologics.

### Chemicals

Poly(ethylene glycol) (PEG)-dithiol (1 kDa) was purchased from Biopharma PEG, 4-arm PEG-maleimide (20 kDa) was purchased from JenKem Technology USA, Laysan Bio, Inc, and Creative PEGWorks, and alginate (71238), trypsin (T7409), α-galactosidase (G8507), cellulase (C1794), and β-galactosidase (G5160) were purchased from MilliporeSigma. Alginate solutions were prepared in ddH₂O by vigorous heating and stirring. Calcium carbonate and dimercaptosuccinic acid (DMSA) were purchased from ACROS Organics, and o-nitrophenyl β-D-galactopyranoside (ONPG) and X-α-Gal were was purchased from Cayman Chemical. Lumefantrine and EnzChek Cellulase Substrate were was purchased from Fisher Scientific, and halofantrine and desbutyl lumefantrine were purchased from MedChemExpress.

### In Vitro LIFT Hydrogel Formation and Characterization

A polymer solution of 0.5% w/v alginate and 4-arm PEG-maleimide (0-10% w/v) was prepared in ddH₂O; to form hydrogels, 60 μL of this solution was cast into 1 mL of crosslinker solution (200 mM CaCl₂), 10 mM PEG-dithiol or DMSA) using a pipette and incubated for 10-20 min at 37° C., 50 RPM. After, the resulting hydrogels were washed with ddH₂O. In some experiments, hydrogels were cast in 0-100% v/v of porcine gastric fluid diluted with ddH₂O. Concentrated stocks of crosslinker were added to porcine gastric fluid to achieve the stated crosslinker concentrations and % v/v gastric fluid.

Mechanical compression tests were performed using an Instron instrument as previously described with modifications. The gauge length was determined with a digital caliper, and displacement was applied at a rate of 0.05 mm/s until 90% strain. Cyclic compression measurement was performed with a displacement rate of 0.05 mm/s and 5 cycles of 90% strain.

To facilitate rheological characterization, pre-crosslinked LIFT hydrogels were prepared by casting a solution of 0.5% w/v alginate/5% w/v 4-arm PEG-maleimide with 15 mM CaCO₃/30 mM glucono-8-lactone into a 100-mm Petri dish. After 1 h incubation at room temperature, hydrogel samples were made using an 8-mm diameter biopsy punch. Oscillatory rheology studies were performed with a Discovery Series Hybrid Rheometer from TA Instruments. Samples were measured using 8-mm parallel plates fully submerged in a 5 mL bath of crosslinker solution (200 mM CaCl₂)/10 mM PEG-dithiol) at 37° C. 8-mm parallel plates (smallest available size) were considered because they would minimize unexposed surface area at the top and bottom faces of the sample, and therefore best represent crosslinking dynamics in vivo. Data was collected for 1 h with a frequency of 10 rad/s and strain of 1%.

### Model Encapsulation and Release

The following model encapsulants were mixed at a 10 mg/mL concentration in either alginate or LIFT polymer solutions: 155-kDa tetramethylrhodamine isothiocyanate-dextran (TRITC-dextran, MilliporeSigma), and 20- and 100-nm fluorescent carboxylated polystyrene nanoparticles (ThermoFisher). Hydrogels were formed as described above, transferred to simulated gastric fluid (SGF: 34 mM NaCl pH 1.77) or simulated intestinal fluid (SIF, Cole-Parmer), and then incubated at 37° C., 50 RPM. The supernatant was sampled at various timepoints with replacement. For each hydrogel and model encapsulant, three separate experiments were performed simultaneously, and release was calculated according to respective standards diluted in either SGF or SIF.

### Cytotoxicity

Cytotoxicity of gel constituents (4-arm PEG-maleimide, PEG-dithiol, DMSA, CaCl₂)) was determined for 4 different cell lines: Caco-2, HT-29, Hepa1-6, and CV-1. Alginate was unable to be tested due to its viscosity and incompatibility with robotic fluid handlers. Cells were plated at 15,000 cells/well in DMEM+10% FBS and incubated overnight. After, cells were incubated in the indicated treatments and concentrations for 24 h in DMEM+10% FBS, and viability was quantified using CellTiter-Glo (Promega) and calculated as a percentage of untreated cells.

### In Vivo LIFT Hydrogel Formation and Characterization

Hydrogels were administered into Yorkshire pigs via endoscopy. To facilitate visualization, gastric fluid was removed. Crosslinker solution (200 mL, 200 mM CaCl₂) and 10 mM DMSA or PEG-dithiol) was first administered, and then the endoscope was purged with air and water. After, 20-40 mL of hydrogel precursor (typically 0.5% w/v alginate/5% w/v 4-arm PEG-maleimide) was similarly administered. In some experiments, the order was reversed. For mechanical characterization, pigs were sacrificed 6-8 h after hydrogel administration, and the hydrogels were retrieved and tested as described above.

To monitor hydrogel retention kinetics in the porcine gastrointestinal tract and for acute toxicity, hydrogels were loaded with barium sulfate (20% w/v) for X-ray imaging, and images were collected immediately after administration, 4-5 h, and on days 1 and 2. Serum was collected before hydrogel administration (baseline) and on days 1 and 2 for metabolic analysis. Throughout, pigs were clinically monitored for gastrointestinal symptoms (e.g., eating cessation, vomiting).

### Ex Vivo LIFT Characterization

LIFT hydrogels were characterized for yield and mechanical properties after formation in a gastric tissue environment or normal plastic plate as a control. To recreate an ex vivo gastric tissue environment, abattoir-sourced porcine stomachs were cut into strips and briefly washed with ddH₂O. Tissue was then applied to a plate and secured with a magnetic device that creates individual wells for experimentation. Crosslinker solution (400 μL, 200 mM CaCl₂/10 mM DMSA or PEG-dithiol) was applied to these wells or the wells of a 48-well plate, and then 50 μL of polymer solution (0.5% alginate/5% w/v 4-arm PEG-maleimide) was drop cast into these wells. After incubation for 20 min at 37° C., 50 RPM, hydrogels were briefly washed with ddH₂O and then weighed. These same hydrogels were mechanically characterized as described above.

To test LIFT hydrogel adhesion to gastric tissue, hydrogels were applied to the center of each well of magnetic device-secured gastric tissue and then incubated for 5 min at 37° C. After, the plate was tilted at ˜45°, and 400 μL ddH₂O was added and then removed from each well. Hydrogel location within the well was recorded before and after washing and tilting.

### Encapsulated Lumefantrine Pharmacokinetics

All animal studies were performed only after MIT CAC review and approval and under veterinarian supervision. Pigs were dosed with the following treatments via endoscopy: free lumefantrine, lumefantrine encapsulated in alginate hydrogels, and lumefantrine encapsulated in LIFT hydrogels (n=3 each). All pigs were dosed with a total of 960 mg lumefantrine. For free lumefantrine, drug powder was weighed and placed across three gelatin capsules. For hydrogel formulations, lumefantrine powder was suspended in polymer solution (0.5% w/v alginate or 0.5% w/v alginate/5% 4-arm PEG-maleimide), mixed, and administered after crosslinker solution (200 mM CaCl₂/10 mM DMSA). Blood was sampled via an installed ear catheter at the indicated time points, and lumefantrine area under the curve was calculated by the trapezoidal rule.

Plasma lumefantrine and desbutyl lumefantrine were separated via high pressure liquid chromatography (HPLC) and quantified with an Agilent 6495A electrospray ionization (ESI) triple quadrupole mass spectrometer (Agilent Technologies, Santa Clara, CA). Samples were introduced to the system with a 5 μL injection volume. Chromatography was performed on an Acquity BEH C18 column (2.1×50 mm, d_{P} 1.8 μm, Waters, Milford, MA), heated to 50° C., with a binary mobile phase composed of 0.1% formic acid in water (v/v, A) and 5% tetrahydrofuran in methanol (v/v, B). The mobile phase was pumped at 0.5 mL/min and gradient programmed as: 0 min, 5% B; 5 min, 95% B. The total method runtime was 7 min and had a 2 min re-equilibration time between injections. For positive ionization ESI source conditions, the iFunnel high pressure radiofrequency was set to 150 V, and low pressure set to 60 V. Nebulizer drying gas temperature was set to 210° C. with a flow rate of 15 L/min at 35 psig. Sheath gas temperature was set to 250° C. with a flow rate of 12 L/min. Nozzle voltage was set to 1500 V and capillary voltage was set to 3500 V. Dynamic multiple reaction-monitoring was used to quantify analytes. Lumefantrine was monitored at transitions 528.16 to 510.00 m/z at 28 collision energy (CE), with a qualifier transition from 528.16 to 383.00 m/z (40 CE). Desbutyl lumefantrine was monitored at 472.1 to 454.1 m/z (20 CE) and qualified from 472.1 to 346 m/z (36 CE). Halofantrine was used as an internal standard and quantified with the 500.18 to 142.10 m/z transition (24 CE) and qualified with the 500.18 to 100.10 m/z transition. All transitions used a cell accelerator voltage of 4. Data analysis was performed with MassHunter B10.1 (Agilent Technologies, Santa Clara, CA). Linear calibration curves were weighted by the reciprocal of the standard concentrations used.

A ten-point calibration curve of halofantrine, lumefantrine and desbutyl lumefantrine was prepared with concentrations ranging from 1 to 2500 ng/mL. For plasma sample preparation, 250 μL of plasma, 20 μL of halofantrine at 2500 ng/mL and 730 μL of 90:10 methanol:tetrahydrofuran was added for protein precipitation. Samples were vortexed and centrifuged at 15,000×g for 15 min. The resulting supernatant (200 μL) was transferred to glass vials for analysis.

Lactase Activity after Dithiol Molecule Treatment

Lactase (18 μg, 60 μL) was added to DMSA or PEG-dithiol to a final DMSA/PEG-dithiol concentration of 2.5, 5, or 10 mM. Treatments were incubated at 37° C. for 20 min. Lactase activity was assayed by adding 60 μL of 5 mM ONPG and incubation for 1 min at room temperature. After, 300 μL of 1 M Na₂CO₃ was added to stop the reaction, and the absorbance of the solution was read at λ=420 nm.

### Lactase Encapsulation in LIFT Hydrogels

The effect of acid on enzyme activity was determined by treating lactase (0.24 mg, 60 μL) with either SGF or PBS and incubating at 37° C., 50 RPM for various times. Enzymatic activity after incubation was determined by adding 60 μL of 5 mM ONPG and incubation for 1 min at room temperature. After, 300 μL of 1 M Na₂CO₃ was added to stop the reaction, and the absorbance of the solution was read at λ=420 nm. For in vitro hydrogel experiments, lactase (0.20 mg) was suspended in 60 μL hydrogel precursor (0.5% w/v alginate/5% 4-arm PEG-maleimide) and cast in crosslinker solution (200 mM CaCl₂/10 mM PEG-SH). Alginate-only hydrogels were prepared in 200 mM CaCl₂ solution only, and both hydrogels were prepared with and without CaCO₃ (42.68 mg/mL). Hydrogels were then challenged with SGF for 1 h at 37° C. After acid incubation, enzymatic activity was quantified as above. For trypsin challenge experiments, lactase-containing hydrogels (60 μL, 0.20 mg lactase) were prepared and incubated with trypsin (40 mg/mL) for 6 h at 37° C. Free lactase and alginate-only hydrogels were included as controls. Lactase enzyme activity was quantified as previously described, and compared between trypsin-treated samples and naive samples to determine relative absorbance.

Encapsulated enzyme activity was tested in rat and porcine models. Rats (>400 g) were fasted overnight prior to administration. The following day, 3 mL of crosslinker solution (200 mM CaCl₂), 10 mM PEG-dithiol) was administered via oral gavage immediately followed by 1 mL of hydrogel precursor (0.5% w/v alginate/5% w/v 4-arm PEG-maleimide with 0.24 mg lactase). Calcium carbonate (42.69 mg) was included either in the crosslinker solution (separate) or in the hydrogel precursor (co-encapsulated). After 1 or 2 h, rats were euthanized, and the hydrogels were collected. Hydrogels were weighed and minced, and enzymatic activity was quantified as described above and normalized by hydrogel mass. Encapsulated enzyme activity was also tested in Yorkshire pigs. Hydrogels were administered via endoscopy: first, 200 mL of crosslinker (200 mM CaCl₂)/10 mM PEG-dithiol) was administered followed by 20 mL of 0.5% w/v alginate/5% w/v 4-arm PEG-maleimide containing lactase (40.45 mg) with or without co-encapsulated CaCO₃ (2 g). After 6 h, hydrogels were retrieved, and lactase activity was quantified as described above.

### Statistical Analysis

For all tests, an α=0.05 was set for statistical significance. Single comparison tests were performed using a Student's t-test, and multiple comparisons were performed using a one-way ANOVA with post-hoc Tukey's multiple comparisons test.

### Example 2

A formulation where probiotics are incorporated into the hydrogel precursor and then encapsulated in situ within the stomach when in contact with the crosslinker solution. Additional excipients may be incorporated to protect bacteria viability within the gastrointestinal tract. See FIGS. 15A-15E.

### Example 2

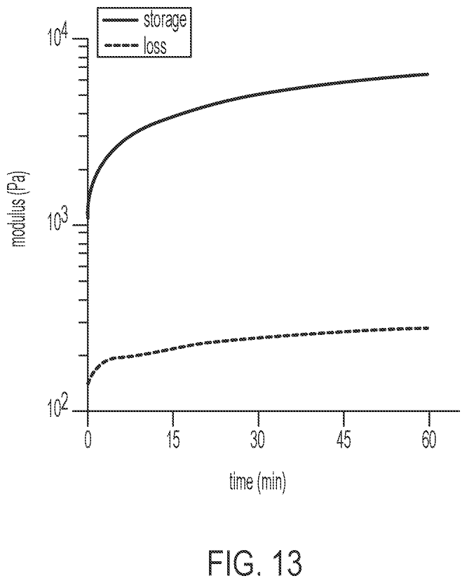

A double network hydrogel was formed by mixing a first composition comprising dithiol-PEG and calcium and a second composition comprising alginate and a 4-arm PEG maleimide. The viscoelastic properties of the double network hydrogel were measured as a function of time after mixing the first and second compositions. The storage and loss moduli as a function of time of the double network hydrogel are shown in FIG. 13.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a first pharmaceutically acceptable fluid; and
a second pharmaceutically acceptable fluid,
wherein the first and second pharmaceutically acceptable fluids are configured to polymerize to form a double network hydrogel when mixed, and
wherein the first and second pharmaceutically acceptable fluids are suitable for oral administration to a subject.

2. A composition as in claim 1,
wherein the first pharmaceutically acceptable fluid comprises at least two crosslinkers; and
the second pharmaceutically acceptable fluid comprises at least two hydrogel precursors.

3. A kit, comprising:
at least 200 mL of the first pharmaceutically acceptable fluid and at least 20 mL of the second pharmaceutically acceptable fluid of claim 1.

4. A method of forming a double network hydrogel at a location internal to a subject, comprising:
orally administering a first pharmaceutically acceptable fluid and a second pharmaceutically acceptable fluid at a location internal of a subject.

5. A method of forming a double network hydrogel within a gastrointestinal tract of a subject, comprising:
polymerizing at least a portion of first pharmaceutically acceptable fluid and at least a portion of second pharmaceutically acceptable fluid within the gastrointestinal tract the subject.

6. The composition, kit, or method as in any preceding claim, wherein the double network hydrogel comprises a first ionically-crosslinked polymer and a second covalently crosslinked polymer.

7. The composition, kit, or method as in any preceding claim, wherein the first ionically-crosslinked polymer comprises a polyanionic polymer.

8. The composition, kit, or method as in any preceding claim, wherein the polyanionic polymer comprises a backbone comprising carboxylic acids.

9. The composition, kit, or method as in any preceding claim, wherein the second covalently-crosslinked polymer comprises polyethylene glycol.

10. The composition, kit, or method as in any preceding claim, wherein the second covalently-crosslinked polymer comprises a maleimide and/or an azide.

11. The composition, kit, or method as in any preceding claim, wherein the double network hydrogel comprises alginates, karaya gum, gellan gum, and/or poly(ethylene glycols).

12. The composition, kit, or method as in any preceding claim, wherein the composition further comprises a therapeutic agent.

13. The composition, kit, or method as in any preceding claim, wherein the composition further comprises an excipient.

14. The composition, kit, or method as in any preceding claim, wherein the double network hydrogel is pharmaceutically acceptable.

15. The composition, kit, or method as in any preceding claim, wherein the first pharmaceutically acceptable fluid comprises calcium and a dithiol crosslinker.

16. The composition, kit, or method as in any preceding claim, wherein the second pharmaceutically acceptable fluid comprises alginate and a 4-arm poly(ethylene glycol)-maleimide.

17. The composition, kit, or method as in any preceding claim, wherein the dithiol crosslinker comprising poly(ethylene glycol)-dithiol and/or dimercaptosuccinic acid.

18. The composition, kit, or method as in any preceding claim, wherein each of the first ionically-crosslinked polymer and the second covalently crosslinked polymer polymerize in less than or equal to 30 minutes.

19. The composition, kit, or method as in any preceding claim, wherein each of the first ionically-crosslinked polymer and the second covalently crosslinked polymer polymerize in less than or equal to 10 minutes.

20. The composition, kit, or method as in any preceding claim, wherein each of the first ionically-crosslinked polymer and the second covalently crosslinked polymer polymerize in less than or equal to 5 minutes.

21. The composition, kit, or method as in any preceding claim, wherein the location internal to the subject is the stomach.

22. A composition comprising:
a fluid drinkable by a subject, formulated to thicken to form a solid excipient when exposed to conditions similar or identical to those of at least one portion of the gastrointestinal tract of the subject.

23. A composition, comprising:
a first configuration having a first, non-solid viscosity and formulated to thicken to a second configuration having a second viscosity greater than the first viscosity when exposed to conditions similar or identical to those of at least one portion of the gastrointestinal tract of a subject.

24. A composition as in claim B, wherein the first configuration is drinkable by a subject, and the second configuration is a spatially-segregated excipient locus internal of the gastrointestinal tract of the subject.

25. A method comprising:
drinking a fluid; and
allowing the fluid to at least partially harden internally of the gastrointestinal tract to form an excipient.

26. A method comprising:
drinking a first fluid;
drinking a second fluid; and
allowing the first and second fluids to at least partially harden internally of the gastrointestinal tract to form an excipient.

27. The method of claim 26, wherein the excipient is a first excipient, and the excipient encapsulates a small molecule and/or a second excipient.

28. The method of claim 26 or 27, wherein allowing the first and second fluids to at least partially harden internally comprises polymerizing at least a first component of the first solution and at least a second component from the second solution.

29. A kit for delivery of a substance to a subject, comprising:
a package containing a first composition and a second composition arranged so as not to be homogenously mixed prior to delivery to the subject, the package configured to deliver at least one of the first and second compositions to a subject as a fluid, wherein the first and second compositions are formulated such that when at least one composition is exposed to conditions similar or identical to those of at least one portion of the gastrointestinal tract of the subject, and at least a portion of the first composition is mixed with at least a portion of the second composition, a solid drug-release excipient is formed.

30. A kit as in any one of claim 29, wherein the first and second compositions are separated from each other in the kit.

31. A kit as in any one of claims 29-30, wherein both the first and second compositions are fluids.

32. A kit as in any one of claims 29-31, wherein the kit is configured to deliver the first and second compositions at non-identical times and/or flow rates to the subject, and to allow at least a portion of the first composition to be mixed with at least a portion of the second composition within the gastrointestinal tract of the subject.

33. A kit as in any one of claims 29-32, wherein the first and second compositions are separated from each other in the kit, and the kit is configured to deliver the first and second compositions sequentially to the subject.

34. A kit as in any one of claims 29-33, wherein the first and second compositions are present in a volume ratio of at least 2:1 relative to each other in the kit is configured to deliver the first and second compositions at non-identical times and/or flow rates to the subject, and to allow at least a portion of the first composition to be mixed with at least a portion of the second composition within the gastrointestinal tract of the subject.

35. A kit as in any one of claims 29-34, wherein the first and second compositions are present in a volume ratio of at least 4:1 relative to each other in the kit.

36. A kit as in any one of claims 29-35, wherein the first and second compositions are separated from each other in the kit, and the kit is configured to deliver the first and second compositions sequentially to the subject.

37. The composition, kit, or method as in any preceding claim, wherein the first and second compositions contain reactants configured to react internal to a subject and harden.

38. The composition, kit, or method as in any preceding claim, wherein the first and second compositions contain reactants configured to react internal to a gastrointestinal tract of a subject to form a hardened hydrogel.

39. The composition, kit, or method as in any preceding claim, wherein the fluid contains reactants that react internally to harden.

40. The composition, kit, or method as in any preceding claim, wherein the reactants are dissolved in fluid (distinguished from particulates in suspension)

41. The composition, kit, or method as in any preceding claim, wherein the fluid can be ingested in at least two portions separated in time or space, with potential overlap, or completely separate.

42. The composition, kit, or method as in any preceding claim, wherein the first fluid composition is a precursor and the second fluid/composition is a crosslinker.

43. The composition, kit, or method as in any preceding claim, wherein the first fluid/composition is a crosslinker and the second fluid/composition is a precursor.

44. The composition, kit, or method as in any preceding claim, wherein the method further comprises the ingesting the first pharmaceutically acceptable fluid and then ingesting the second pharmaceutically acceptable fluid.

45. The composition, kit, or method as in any preceding claim, wherein the second configuration is an excipient.

46. The composition, kit, or method as in any preceding claim, wherein the second configuration is a double network hydrogel.

47. The composition, kit, or method as in any preceding claim, wherein the first configuration is a fluid comprising a reactant and/or a crosslinker.

48. The composition, kit, or method as in any preceding claim, wherein a first viscosity is no more than 100,000 mPA s when measured at a temperature of 25 degrees C.

49. The composition, kit, or method as in any preceding claim, wherein a second viscosity is at least 1 mPA s when measured at a temperature of 25 degrees C.

50. The composition, kit, or method as in any preceding claim, further comprising an active agent in the first and/or second fluid.

51. A hydrogel that undergoes a transition from a liquid-to-solid state within the stomach and comprises two distinct polymer networks.

52. A kit comprising two solutions, wherein the two solutions, when mixed at a location internal to a subject, polymerize to a solid state comprising two distinct polymer networks, wherein a first polymer network comprises an ionic bond and a second polymer network comprises a covalent bond.

53. The kit of claim 52, wherein the location internal to the subject is the stomach.

54. The two polymer networks of claim 52 or 53, wherein the first polymer network comprises alginate and calcium and the second polymer network comprises a 4-arm poly(ethylene glycol)-maleimide and a dithiol crosslinker comprising poly(ethylene glycol)-dithiol and/or dimercaptosuccinic acid.

55. The kit of any one of claims 51-54, wherein a first of the two solutions is a crosslinking solution consisting of FDA-approved and/or generally recognized as safe dithiol-containing molecules.

56. The kit of any one of claims 51-55, wherein a first of the two solutions is a crosslinking solution comprises dithiol-containing poly(ethylene glycol), and/or dimercaptosuccinic acid.

57. A method, comprising:
ingesting a first fluid comprising a crosslinker;
after ingesting the first fluid, ingesting a second fluid comprising a hydrogel; and
forming discrete hydrogels having an average maximum dimension of less than or equal to 10 cm.

58. A method, comprising:
ingesting a first fluid comprising a hydrogel precursor;
after ingesting the first fluid, ingesting a second fluid comprising a crosslinker; and
forming discrete hydrogels having an average maximum dimension of less than or equal to 10 cm.

59. The method of claim 57 or 58, wherein the first fluid is imbibed at a volume of at least 200 mL.

60. The first fluid of any one of the preceding claims, further comprising a small molecule, a probiotic, a protein, an excipient, a multi-arm PEG functionalized with a reactive end-group, and/or divalent cation-crosslinked polymers such as alginate.

61. The second fluid of any one of the preceding claims, further comprising a small molecule, a probiotic, a protein, an excipient, a multi-arm PEG functionalized with a reactive end-group, and/or divalent cation-crosslinked polymers such as alginate.

62. The hydrogel precursor of any one of the preceding claims, wherein the multi-arm PEG comprises 2 arms, 4 arms, and/or 8 arms

63. The hydrogel solution of any one of the preceding claims, wherein the multi-arm PEG comprises a maleimide reactive end-group.

64. The hydrogels of any of the preceding claims, wherein a shape and/or size of the hydrogel is compatible with gastric clearance.

65. A kit, composition, or method as in any preceding claim, further comprising a delivery device comprising a first receptacle configured to contain the first fluid, a second receptacle configured to contain the second fluid, and at least one delivery pathway configured to deliver the first and second fluids to a location externally or internally of a subject.

66. A kit, composition, or method as in claim 65, wherein the delivery device includes at least one fluid pathway configured to bring at least a portion of each of the first and second fluids into contact with the other prior to delivery of a combined fluid to a location externally or internally of a subject.

67. A kit, composition, or method as in any preceding claim, wherein the composition or fluid is suitable for oral administration to the subject.
